(19) United States
(12) Patent Application Publication
(10) Pub. No.: US 2013/0045483 A1
(43) Pub. Date: Feb. 21, 2013

(54) YEAST CELLS EXPRESSING AMYLOID BETA AND USES THEREFOR

(71) Applicants: SEBASTIAN TREUSCH, Lawrenceville, NJ (US); KENT E.S. MATLACK, BROOKLINE, MA (US); SUSAN L. LINDQUIST, Cambridge, MA (US)

(72) Inventors: SEBASTIAN TREUSCH, Lawrenceville, NJ (US); KENT E.S. MATLACK, BROOKLINE, MA (US); SUSAN L. LINDQUIST, Cambridge, MA (US)

(73) Assignee: WHITEHEAD INSTITUTE FOR BIOMEDICAL RESEARCH, CAMBRIDGE, MA (US)

(21) Appl. No.: 13/538,089

(22) Filed: Jun. 29, 2012

Related U.S. Application Data
(60) Provisional application No. 61/504,052, filed on Jul. 1, 2011.

(51) Int. Cl. C12N 5/071; C12Q 1/68; C40B 30/06; C12N 1/19; C12N 5/10

(52) U.S. Cl. 435/6.13; 435/254.2; 435/375; 435/325; 435/366; 506/10

(57) ABSTRACT

Disclosed are yeast cells expressing a polypeptide comprising a signal sequence and a human amyloid beta protein. Also disclosed are methods of screening yeast cells to identify compounds that prevent or suppress amyloid beta-induced toxicity and genetic suppressors or enhancers of amyloid beta-induced toxicity. Compounds identified by such screens can be used to treat or prevent neurodegenerative disorders such as Alzheimer's disease.

### CROSS REFERENCE TO RELATED APPLICATIONS

This application claims priority from U.S. Provisional Application No. 61/504,052, filed Jul. 1, 2011. The entire content of the prior application is incorporated herein by reference in its entirety.

### STATEMENT AS TO FEDERALLY SPONSORED RESEARCH

This invention was made with government support under grant numbers GM25874-25 NIH and NSO60957 awarded by the National Institutes of Health. The Government has certain rights in the invention.

### TECHNICAL FIELD

The invention relates to protein chemistry and cellular and molecular biology

### BACKGROUND

Alzheimer's disease is a neurodegenerative disorder characterized by neurofibrillary tangles and plaques containing an amyloid beta peptide. Patients with Alzheimer's disease exhibit progressive dementia and personality dysfunction. Proteolytic cleavage of the amyloid precursor protein (APP) results in the generation of an amyloid beta peptide having a length ranging from 38 to 43 amino acids. The amyloid beta 1-42 peptide is particularly prone to self-aggregation and is strongly linked to development of Alzheimer's disease.

### SUMMARY

The invention is based, at least in part, on the discovery that a fusion polypeptide containing a signal sequence and a human amyloid beta protein is toxic when expressed in a yeast cell. This discovery permits the carrying out of screening assays using amyloid beta-expressing yeast cells to identify compounds or genetic factors that modulate amyloid beta-induced toxicity. Compounds identified by such screens can be used for the treatment or prevention of neurodegenerative diseases such as Alzheimer's disease.

Described herein is a yeast cell comprising an expression construct comprising a promoter operably linked to a nucleic acid encoding a polypeptide comprising a signal sequence and a human amyloid beta protein, wherein expression of the nucleic acid and production of the polypeptide in the cell results in a decrease in growth or viability of the cell. In some embodiments, expression of the nucleic acid and production of the polypeptide renders the yeast cell non-viable.

A signal sequence causes a polypeptide to be targeted to the endoplasmic reticulum within a cell. In some embodiments, the signal sequence is located at the amino terminus of the polypeptide encoded by the expression construct. In some embodiments, the signal sequence is one that directs co-translational transport of the encoded polypeptide.

The signal sequence can be identical to a naturally occurring signal sequence or can be an artificial (non-naturally occurring) signal sequence. In some embodiments, the signal sequence is identical to the signal sequence of a naturally occurring yeast protein (e.g., identical to the yeast Kar2p signal sequence). In some embodiments, the signal sequence is identical to the signal sequence of a naturally occurring mammalian protein (e.g., a human protein).

The term “human amyloid beta protein” includes naturally occurring wild type amyloid beta peptides as well as naturally occurring mutant amyloid beta peptides. Wild type amyloid beta peptides include amyloid beta 1-38, amyloid beta 1-39, amyloid beta 1-40, amyloid beta 1-41, amyloid beta 1-42, and amyloid beta 1-43. Amyloid beta mutations include A2T, H6R, D7N, A21G, E22G (Arctic), E22Q (Dutch), E22K (Italian), D23N (Iowa), A42T, and A42V (wherein the numbering is relative to the amyloid beta peptide of SEQ ID NO:3). These mutations may optionally be present in any of the amyloid beta peptides 1-38, 1-39, 1-40, 1-41, 1-42, and 1-43.

In alternate embodiments, a variant of a human amyloid beta protein can be used. A “variant human amyloid beta protein” differs (via substitution, deletion, and/or insertion) from a naturally occurring amyloid beta peptide at up to 10 amino acids (e.g., differs at no more than 5 amino acids, differs at no more than 4 amino acids, differs at no more than 3 amino acids, differs at no more than 2 amino acids, or differs at 1 amino acid) and retains the ability to cause a decrease in growth or viability of a yeast cell when expressed in a fusion polypeptide described herein.

In some embodiments, the signal sequence is identical to the signal sequence of a naturally occurring yeast protein (e.g., the signal sequence is identical to the yeast Kar2p signal sequence) and the human amyloid beta protein is wild type amyloid beta 1-42. For example, the polypeptide can comprise or consist of the amino acid sequence of SEQ ID NO:1. Exemplary nucleic acids encoding the amino acid sequence of SEQ ID NO:1 include SEQ ID NO:2 and nucleotides 33 to 284 of SEQ ID NO:2.

In some embodiments, the polypeptide encoded by an expression construct described herein is no more than 150, 125, or 100 amino acids in length.

In some embodiments, the human amyloid beta protein portion of the polypeptide encoded by an expression construct described herein is no more than 75, 50, or 45 amino acids in length.

In some embodiments, the polypeptide encoded by an expression construct described herein consists of a signal sequence and a human amyloid beta protein. In some embodiments, the polypeptide encoded by an expression construct described herein consists of a signal sequence, a linker peptide sequence, and a human amyloid beta protein.

In some embodiments, cleavage of the human amyloid beta protein portion of the polypeptide may occur before translation of the entire polypeptide is complete. This phenomenon is encompassed by the phrase “production of the polypeptide in the cell results in a decrease in growth or viability of the cell,” so long as at least the human amyloid beta protein portion of the polypeptide is translated and results in a decrease in growth or viability of the cell.

In addition to a signal sequence and a human amyloid beta protein, the polypeptide encoded by an expression construct described herein can also contain one or more heterologous peptide sequences, such as an expression tag. A heterologous peptide sequence can be present at the amino terminus of the polypeptide, between the signal sequence and the human amyloid beta protein, and/or at the carboxy terminus of the polypeptide.

An expression construct described herein can optionally be integrated in the genome of the yeast cell. For example, the expression construct can be an integrative plasmid such as pRS303, pRS304, pRS305, pRS306, or a derivative thereof. A yeast cell can have one or more (e.g., at least two, at least three, or at least four) copies (e.g., integrated copies) of an expression construct.

The promoter can be an inducible promoter such as GAL1-10, GAL1, GALL, GALS, GPD, ADH, TEF, CYC1, MRP7, MET25, TET, VP16, or VP16-ER. Alternatively, the promoter can be a constitutively active promoter.

The polypeptide can be a fusion protein containing a detectable protein (e.g., a fluorescent protein, an enzyme, or an epitope). Exemplary fluorescent proteins include red fluorescent protein, green fluorescent protein, blue fluorescent protein, yellow fluorescent protein, and cyan fluorescent protein.

In some embodiments, the yeast is Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., or Geotrichum fermentans.

In some embodiments, at least one gene that encodes a protein involved in drug efflux or cell permeability is disrupted in the yeast cell. For example, one or more of the genes PDR1, PDR3, PDR5, SNQ2, or ERG6 can be disrupted in the yeast cell.

Also disclosed is a method of inducing toxicity in a yeast cell by: providing a yeast cell described herein; and inducing a level of expression of the nucleic acid in the yeast cell that is toxic to the yeast cell.

Also disclosed is a method of identifying a compound that prevents or suppresses amyloid beta-induced toxicity by: culturing a yeast cell described herein in the presence of a candidate agent and under conditions that allow for expression of the nucleic acid at a level that, in the absence of the candidate agent, is sufficient to induce toxicity in the cell; measuring cell growth or viability in the presence of the candidate agent; and comparing cell growth or viability measured in the presence of the candidate agent to cell growth or viability in the absence of the candidate agent, wherein if cell growth or viability is increased in the presence of the candidate agent as compared to in the absence of the candidate agent, then the candidate agent is identified as a compound that prevents or suppresses amyloid beta-induced toxicity.

Also disclosed is a method of identifying a genetic suppressor or enhancer of amyloid beta-induced toxicity by: providing a yeast cell described herein, wherein the yeast cell has been genetically modified to overexpress a gene; culturing the yeast cell under conditions that allow for expression of the protein at a level that, in the absence of overexpression of the gene, is sufficient to induce toxicity in the yeast cell; measuring cell growth or viability in the presence of overexpression of the gene; and comparing cell growth or viability measured in the presence of overexpression of the gene to cell growth or viability in the absence of overexpression of the gene, wherein (i) if cell growth or viability is increased in the presence of overexpression of the gene as compared to in the absence of overexpression of the gene, then the gene is identified as a genetic suppressor of amyloid beta-induced toxicity, and (ii) if cell growth or viability is decreased in the presence of overexpression of the gene as compared to in the absence of overexpression of the gene, then the gene is identified as a genetic enhancer of amyloid beta-induced toxicity.

Also disclosed are certain yeast genes identified according to the foregoing method, and (for some of these genes) counterparts thereof found in other organisms (e.g., counterparts present in mammalian species such as humans), and their encoded proteins. Also disclosed are cells that express a toxicity-inducing amount or form of amyloid beta protein, wherein the cells overexpress a genetic suppressor or enhancer of amyloid beta-induced toxicity. For example, disclosed are yeast cells that express a fusion polypeptide containing a signal sequence and a human amyloid beta protein, wherein the yeast cells overexpress an identified genetic suppressor or enhancer of amyloid beta-induced toxicity. Further disclosed are methods, e.g., screening assays and methods of treatment, relating to the identified genetic enhancers and suppressors.

In some aspects, the invention provides a method of modulating amyloid beta-mediated toxicity, the method comprising contacting a cell expressing a toxicity-inducing amount or form of amyloid beta with an effective amount of a compound that modulates expression or activity of SPO7, KAR9, POG1, KEM1, XRN1, ROM1, NET1, MID2, BOP3, PMT2, POMT2, PSK1, PASK, YBL061C, SEL1L2, PET111, SLS1, SVL3, IVY1, MVP1, SNX8, PBS2, MAP2K4, PKC1, PKN2, WHI5, SLF1, LARP1, YBL086C, YAP1802, PICALM, YPL014W, RTS1, PPP2R5C, SPT21, FMP48, MARK4, PPR1, TEC1, TEAD2, ADE12, ADSSL1, CRM1, XPO1, NAB3, SLA1, SH3KBP1, RTG3, MITF, SRO9, LARP1B, MBP1, DAPK1, MUM2, INP52, SYNJ1, FCY21, GRR1, FBXL2, VPS9, RABGEF1, or OPY1.

In some aspects, the invention provides a method of inhibiting amyloid beta-mediated toxicity, the method comprising contacting a cell expressing a toxicity-inducing amount or form of amyloid beta with an effective amount of a compound that inhibits expression or activity of XRN1, POMT2, SNX8, MAP2K4, PASK, NET1, SEL1L2, or PKN2 or an effective amount of a compound that enhances expression or activity of PICALM, PPP2R5C, ADSSL1, XPO1, SH3KBP1, SYNJ1, FBXL2, RABGEF1, MARK4, TEAD2, MITF, DAPK1, LARP1, or LARP1B.

Also disclosed is a method of identifying a genetic suppressor or enhancer of amyloid beta-induced toxicity by: providing a yeast cell described herein, wherein an endogenous gene of the yeast cell has been disrupted; culturing the yeast cell under conditions that allow for expression of the protein at a level that, in the absence of disruption of the endogenous gene, is sufficient to induce toxicity in the yeast cell; measuring cell growth or viability in the presence of disruption of the endogenous gene; and comparing cell growth or viability measured in the presence of disruption of the endogenous gene to cell growth or viability in the absence of disruption of the endogenous gene, wherein (i) if cell growth or viability is increased in the presence of disruption of the endogenous gene as compared to in the absence disruption of the endogenous gene, then the gene is identified as a genetic enhancer of amyloid beta-induced toxicity, and (ii) if cell growth or viability is decreased in the presence of disruption of the endogenous gene as compared to in the absence disruption of the endogenous gene, then the gene is identified as a genetic suppressor of amyloid beta-induced toxicity. As an alternative to use of a yeast cell that has had an endogenous gene disrupted, the method may be performed using a yeast cell wherein expression of the endogenous gene is suppressed by use of RNA interference.

In some embodiments, of any of the methods of identifying yeast genes or identifying compounds in yeast cells, the yeast cells are cultured under at least two different culture conditions. In some embodiments, the culture conditions result in different levels of mitochondrial respiration. In some embodiments, the culture conditions comprise use of culture medium comprising glucose, galactose, or glycerol as a carbon source. In some embodiments, yeast cells are cultured under at least three different culture conditions, and a gene is identified as a genetic enhancer or suppressor of amyloid-beta induced toxicity or a compound is identified as a modulator of amyloid beta mediated toxicity (e.g., an inhibitor of amyloid beta toxicity) under at least two of the culture conditions, e.g., under three different culture conditions or all of the culture conditions tested.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a series of photographs of yeast cells transformed with either an empty vector (top row of each photograph) or a galactose-inducible expression plasmid encoding a yeast Kar2p signal sequence/human amyloid beta 1-42 fusion polypeptide (bottom row of each photograph). The yeast cells were spotted on glucose or galactose and growth was assessed. The experimental and control transformants grew equally well on the glucose plates (“unexpressed”), whereas amyloid beta expression profoundly inhibited cell growth (“expressed”) in a copy number-dependent manner.

FIG. 2 is a three dimensional graph depicting the results of a screen to identify compounds that suppress toxicity induced by expression of a yeast Kar2p signal sequence/human amyloid beta 1-42 fusion polypeptide.

FIG. 3: Further analysis of expression of yeast Kar2p signal sequence/human amyloid beta 1-42 fusion polypeptide (also referred to herein as “ssAβ 1-42”) in yeast. (A) Comparison of yeast Kar2p signal sequence/human amyloid beta 1-42 fusion polypeptide (also referred to herein as “ssAβ 1-42”) to yeast Kar2p signal sequence/human amyloid beta 1-40 fusion polypeptide (also referred to herein as “ssAβ 1-40”), ssBPTI (WT and C51A) and Pdi1. ssAβ 1-42 was more toxic than ssAβ 1-40 and other proteins targeted to the secretory pathway in the same manner as ssAβ. Proteins were expressed using the inducible GAL promoter and a high copy number plasmid. Strains carrying the plasmids were serially diluted and spotted on inducing (Galactose) and non-inducing (Glucose) media. (B) Construction of ssAβ 1-42 strains used in genetic screen. Expression of ssAβ 1-42 from a single integrated genomic copy was not toxic, but integration of several copies of the construct resulted in robust toxicity. (C) Aβ 1-42 expression in these strains was detected by immunoblot analysis using the 6E 10 Aβ antibody. Longer exposure revealed the formation of higher molecular weight species consistent with the formation of Aβ oligomers and dimers. (D) Immunostaining for ssAβ 1-42 reveals localization to the endoplasmic reticulum (ER). Aβ was detected in the ER (ring surrounding the nucleus stained blue using DAPI) and in small foci throughout the cell (arrowheads). These foci may represent Aβ aggregates or accumulation in vesicles.

FIG. 4: Suppression of Aβ toxicity by clioquinol. Addition of clioquinol to a ssAβ 1-42 strain grown in inducing media rescued growth levels in a dose dependent manner. At the optimal concentration clioquinol restored growth levels to those of a control strain expressing YFP. At higher concentrations clioquinol became toxic to both strains.

FIG. 5: Localization of Abeta to the ER. ssAbeta was expressed using GAL inducible high copy number plasmid in the indicated GFP-tagged strains. Co-immunostaining for Sec61-GFP and Abeta shows co-localization of Abeta with an ER marker. Sec13 and Sec21 are markers for ER- Golgi vesicles. Abeta shows weak co-localization with Sec21, but no co-localization with Sec13.

FIG. 6: shows an example of screening plates used to identify genetic enhancers and suppressors of ssAβ toxicity. Transformants were spotted 4 times using a Singer RoToRrobot. Enhancers resulted in decreased growth and suppressors in increased growth. Empty quadrants on the plate are the result of empty wells in the plasmid library.

FIG. 7: Effect of suppressors on YFP expression levels. Putative suppressors were transformed into strain carrying an integrated GAL inducible YFP construct. The effect of putative hits on YFP expression levels was determined after overnight growth in the indicated media using flow cytometry. Hits that significantly decreased the levels of YFP in comparison to the vector control were eliminated as false positives (e.g., Haal). Suppressors that showed activity on all three media were retained even if they lowered YFP levels on one of the growth conditions.

FIG. 8: Effect of hits with human homologs. Strains expressing ssAbeta and the indicated modifiers were grown for 8 h or 16 h in inducing media. Cells were lysed and immunoblotted for Abeta using the 6E10 antibody and for Pgk1 as a loading control. The hits did not alter Abeta levels significantly. Although subtle differences were observed between strains these did not prove to be reproducible.

FIG. 9: Effect of hits with human homologs on Abeta localization. Strains expressing ssAbeta and the modifiers with human homologs were grown for 8 h in inducing media and immunostained for Abeta. Most modifiers had no effect on Abeta localization. However, Pbs2 (yeast Map2K4) enhanced the formation of Abeta foci/vesicles.

FIG. 10: Aβ 1-42 causes defects in endocytosis and protein trafficking. (A) Expression of ssAβ 1-42 in a strain with GFP-tagged clathrin light chain (Clcl-GFP) and effects of co-expression of YAP1802 or INP52. (B) Aβ 1-42 resulted in a defect in Ste3-YFP trafficking. In the control strain Ste3-YFP was trafficked to the vacuole, in contrast Aβ expression resulted in the accumulation of Ste3-YFP in cytoplasmic foci. Coexpression of YAP1802, INP52 and SLA1 suppressed this effect of Aβ 1-42 (see FIG. 12 for the latter). Scale bars are 5 μm and all figures are on the same scale.

FIG. 11: Aβ toxicity modifiers modulate the Aβ-induced change in clathrin localization. A) Images showing effect of expression of ssAβ 1-42 in a strain carrying a haploid clathrin light chain GFP fusion (Clcl-GFP). ssAβ 1-42 expression resulted in an increase in the number and brightness of Clcl-GFP foci, while also decreasing their size. B) Images showing the effects of the indicated suppressors and enhancers with annotated functions in endocytosis and trafficking on the Aβ-induced change in Clcl-GFP localization. Scale bar is 5 μM and all figures are on the same scale.

FIG. 12: Aβ toxicity modifiers modulate the Aβ-induced change in Ste3-YFP trafficking. Images show effect of ssAβ 1-42 expression on Ste3-YFP localization and the effect of indicated modifiers involved in endocytosis and protein trafficking. Scale bar is 5 μM and all figures are on the same scale.

### DETAILED DESCRIPTION

The amyloid beta-expressing yeast cells described herein can be used to identify compounds or genetic factors that modulate amyloid beta-induced toxicity. Compounds identified by such screens can be used for the treatment or prevention of neurodegenerative diseases such as Alzheimer's disease or other diseases characterized by accumulation of amyloid-beta.

### Proteins and Nucleic Acids

Described herein are compositions and methods for identifying candidate compounds that prevent or suppress amyloid beta-induced toxicity and genetic suppressors or enhancers of amyloid beta-induced toxicity. A fusion polypeptide used in the compositions and methods described herein contains a signal sequence and a human amyloid beta protein.

As used herein, the term “human amyloid beta protein” refers to a sequence identical to a naturally occurring 38-43 amino acid amyloid beta peptide that is derived via proteolytic processing of the human amyloid precursor protein (APP) and is associated with amyloid pathologies. The term includes naturally occurring wild type amyloid beta peptides as well as naturally occurring mutant amyloid beta peptides. Wild type amyloid beta peptides include amyloid beta 1-38, amyloid beta 1-39, amyloid beta 1-40, amyloid beta 1-41, amyloid beta 1-42, and amyloid beta 1-43. Amyloid beta mutations include A2T, H6R, D7N, A21G, E22G (Arctic), E22Q (Dutch), E22K (Italian), D23N (Iowa), A42T, and A42V (wherein the numbering is relative to the amyloid beta peptide of SEQ ID NO:3). These mutations may optionally be present in any of the amyloid beta peptides 1-38, 1-39, 1-40, 1-41, 1-42, and 1-43.

Amino acids 1-43 of human amyloid beta, which amino acids are used as the backbone of the amyloid beta peptides described herein, are as follows: DAEFRHDSGYEVHHQKLVFFAEDVGSNKGAIIGLMVGGVVIAT (SEQ ID NO:3).

As used herein, the term “signal sequence” refers to a peptide sequence that is present within a polypeptide and causes the polypeptide to be targeted to the endoplasmic reticulum within a cell. An exemplary signal sequence described in the working examples is the yeast Kar2p signal sequence. However, a wide variety of signal sequences are known and can be used to cause endoplasmic reticulum targeting of the fusion polypeptides described herein. Signal sequences are reviewed in e.g., Wilkinson et al. (1997) J Membr Biol. 155(3):189-97, Haguenauer-Tsapis (1992) Mol. Microbiol. 6(5):573-9, and Pool (2005) Mol Membr Biol. 22(1-2):3-15.

A polypeptide containing a signal sequence and a human amyloid beta protein may optionally be fused with a second domain. The second domain of the fusion protein can optionally be an immunoglobulin element, a dimerizing domain, a targeting domain, a stabilizing domain, or a purification domain. Alternatively, an amyloid beta protein can be fused with a heterologous molecule such as a detection protein. Exemplary detection proteins include: a fluorescent protein such as green fluorescent protein (GFP), cyan fluorescent protein (CFP) or yellow fluorescent protein (YFP); an enzyme such as β-galactosidase or alkaline phosphatase (Aβ); and an epitope such as glutathione-S-transferase (GST) or hemagglutinin (HA). To illustrate, an amyloid beta protein can be fused to GFP at the N- or C-terminus or other parts of the amyloid beta protein. These fusion proteins provide methods for rapid and easy detection and identification of the amyloid beta protein in the recombinant yeast cell.

Also described herein are methods of preparing and transferring nucleic acids encoding an amyloid beta protein into a cell so that the cell expresses the amyloid beta protein. The term “amyloid beta nucleic acid” encompasses a nucleic acid containing a sequence encoding any of the amyloid beta proteins described herein. Exemplary amyloid beta nucleic acids include those encoding amyloid beta 1-42.

The term “nucleic acid” generally refers to at least one molecule or strand of DNA, RNA or a derivative or mimic thereof, containing at least one nucleobase, for example, a naturally occurring purine or pyrimidine base found in DNA or RNA. Generally, the term “nucleic acid” refers to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

### Yeast Cells

Yeast strains that can be used in the compositions and methods described herein include, but are not limited to, Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans. Although much of the discussion herein relates to Saccharomyces cerevisiae which ectopically expresses an abnormally processed protein, this is merely for illustrative purposes. Other yeast strains can be substituted for S. cerevisiae.

Certain aspects of the disclosure relate to screening methods for identifying candidate therapeutic agents (e.g., pharmaceutical, chemical, or genetic agents). The methods described herein can optionally be carried out in yeast strains bearing mutations in the ERG6 gene, the PDR1 gene, the PDR3 gene, the PDR5 gene, the SNQ2 gene, and/or any other gene which affects membrane efflux pumps and/or increases permeability for drugs.

A nucleic acid encoding a fusion polypeptide described herein may be transfected into a yeast cell using nucleic acid vectors that include, but are not limited to, plasmids, linear nucleic acid molecules, artificial chromosomes, and episomal vectors.

Three well known systems used for recombinant plasmid expression and replication in yeast cells include integrative plasmids, low-copy-number ARS-CEN plasmids, and high-copy-number 2μ plasmids. See Sikorski, “Extrachromosomal cloning vectors of Saccharomyces cerevisiae,” in Plasmid, A Practical Approach, Ed. K. G. Hardy, IRL Press, 1993; and Yeast Cloning Vectors and Genes, Current Protocols in Molecular Biology, Section II, Unit 13.4, Eds., Ausubel et al., 1994.

An example of the integrative plasmids is YIp, which is maintained at one copy per haploid genome, and is inherited in Mendelian fashion. Such a plasmid, containing a gene of interest, a bacterial origin of replication and a selectable gene (typically an antibiotic-resistance marker), is produced in bacteria. The purified vector is linearized within the selectable gene and used to transform competent yeast cells.

An example of the low-copy-number ARS-CEN plasmids is YCp, which contains the autonomous replicating sequence (ARS1) and a centromeric sequence (CEN4). These plasmids are usually present at 1-2 copies per cell. Removal of the CEN sequence yields a YRp plasmid, which is typically present in 100-200 copies per cell. However, this plasmid is both mitotically and meiotically unstable.

An example of the high-copy-number 2μ plasmids is YEp, which contains a sequence approximately 1 kb in length (named the 2μ sequence). The 2μ sequence acts as a yeast replicon giving rise to higher plasmid copy number. However, these plasmids are unstable and require selection for maintenance. Copy number is increased by having on the plasmid a selection gene operatively linked to a crippled promoter.

A wide variety of plasmids can be used in the compositions and methods described herein. In one embodiment, the plasmid is an integrative plasmid (e.g., pRS303, pRS304, pRS305, pRS306, or a derivative thereof). See, e.g., Alberti et al. (2007) “A suite of Gateway cloning vectors for high-throughput genetic analysis in Saccharomyces cerevisiae” Yeast 24(10):913-19. In further embodiments, the plasmid is an episomal plasmid (e.g., p426GPD, p416GPD, p426TEF, p423GPD, p425GPD, p424GPD or p426GAL).

Regardless of the type of plasmid used, yeast cells are typically transformed by chemical methods (e.g., as described by Rose et al., 1990, Methods in Yeast Genetics, Cold Spring Harbor Laboratory Press, Cold Spring Harbor, N.Y.). The cells are typically treated with lithium acetate to achieve transformation efficiencies of approximately 10⁴ colony-forming units (transformed cells)/μg of DNA. Yeast perform homologous recombination such that the cut, selectable marker recombines with the mutated (usually a point mutation or a small deletion) host gene to restore function. Transformed cells are then isolated on selective media. Of course, any suitable means of introducing nucleic acids into yeast cells can be used.

The yeast vectors (plasmids) described herein typically contain a yeast origin of replication, an antibiotic resistance gene, a bacterial origin of replication (for propagation in bacterial cells), multiple cloning sites, and a yeast nutritional gene for maintenance in yeast cells. The nutritional gene (or “auxotrophic marker”) is most often one of the following: 1) TRP1 (Phosphoribosylanthranilate isomerase); 2) URA3 (Orotidine-5′-phosphate decarboxylase); 3) LEU2 (3-Isopropylmalate dehydrogenase); 4) HIS3 (Imidazoleglycerolphosphate dehydratase or IGP dehydratase); or 5) LYS2 (α-aminoadipate-semialdehyde dehydrogenase).

The yeast vectors (plasmids) described herein may also contain promoter sequences. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively linked” and “operatively positioned” mean that a promoter is in a correct functional location and/or orientation in relation to a nucleic acid sequence to control transcriptional initiation and/or expression of that sequence.

A promoter may be one naturally associated with a nucleic acid sequence, as may be obtained by isolating the 5′ non-coding sequences located upstream of the coding segment and/or exon. Such a promoter can be referred to as “endogenous.” Alternatively, a promoter may be a recombinant or heterologous promoter, which refers to a promoter that is not normally associated with a nucleic acid sequence in its natural environment. Such promoters may include promoters of other genes and promoters not “naturally occurring.” The promoters employed may be either constitutive or inducible.

For example, various yeast-specific promoters (elements) may be employed to regulate the expression of a RNA in yeast cells. Examples of inducible yeast promoters include GAL1-10, GALL, GALL, GALS, TET, VP16 and VP16-ER. Examples of repressible yeast promoters include Met25. Examples of constitutive yeast promoters include glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), and MRP7. Autonomously replicating expression vectors of yeast containing promoters inducible by glucocorticoid hormones have also been described (Picard et al., 1990), including the glucocorticoid responsive element (GRE). These and other examples are described in Mumber et al., 1995; Ronicke et al., 1997; Gao, 2000, all incorporated herein by reference. Yet other yeast vectors containing constitutive or inducible promoters such as alpha factor, alcohol oxidase, and PGH may be used. For reviews, see Ausubel et al. and Grant et al., 1987. In some embodiments, a yeast strain is used that allows for expression, e.g., inducible expression, from GAL promoters on carbon sources other than galactose. In some embodiments, the strain carries an integrated or episomal (e.g., plasmid-borne) gene encoding a fusion protein, wherein the Gal4 DNA binding domain is fused to a transcriptional activation domain and a regulatory domain. The fusion protein is characterized in that its ability to activate transcription is regulated by binding of a small molecule to the regulatory domain. For example, in some embodiments, the fusion protein does not activate transcription in the absence of the small molecule, whereas in the presence of the small molecule, the fusion protein activates transcription. Exemplary small molecules include, e.g., steroid hormones, wherein the corresponding regulatory domain comprises at least a portion of a receptor for the small molecule. For example, the small molecule may be an estrogen (e.g., estradiol), or analog thereof (e.g., tamoxifen), and the corresponding regulatory domain comprises at least a portion of the estrogen receptor (ER). Exemplary activation domains include, e.g., viral protein activation domains such as the herpes simplex virus protein VP16 activation domain. In some embodiments, the strain carries an integrated or episomal (e.g., plasmid-borne) gene encoding a Gal4-ER-VP16 fusion protein. Presence of an estrogen receptor ligand, e.g., estradiol, in the medium, allows for expression from GAL promoters on carbon sources other than galactose. One of skill in the art will appreciate that numerous ways exist to render expression of a molecule of interest, e.g., an amyloid beta peptide, conditional, e.g., on culture media containing galactose or other carbon sources.

### Modulators of Amyloid Beta-Mediated Toxicity

It has been found that overexpression of certain genes results in a modulation of amyloid beta mediated cellular toxicity. Compounds that modulate expression of these genes or activity of the encoded proteins can be used to modulate amyloid beta mediated toxicity. Modulation, e.g., suppression or enhancement of toxicity, can have a variety of different uses. In some aspects of the invention, compounds that modulate expression of these genes or activity of the encoded proteins can be used to inhibit amyloid beta mediated toxicity and used to treat or prevent diseases associated with such toxicity, e.g., neurodegenerative diseases such as Alzheimer's disease. In some embodiments, compounds that modulate expression of these genes or activity of the encoded proteins can be used to inhibit deleterious effects caused by excessive amyloid beta. Such excessive amyloid beta can result from, e.g., overexpression of the gene that encodes APP. Overexpression of the APP gene can result from e.g., excess copies of the gene (e.g., as a result of trisomy 21), mutations in regulatory regions of the APP gene or in genes that encode regulators of APP expression, etc. Enhancement of amyloid beta-mediated toxicity may be useful, e.g., to facilitate further screening for genes or compounds that inhibit amyloid beta-mediated toxicity and/or in the development of additional model systems for the identification or characterization of potential therapeutic agents for amyloid beta mediated disorders.

As detailed in the accompanying examples, a number of genes have been identified that modulate (e.g., suppress or enhance) cellular toxicity associated with overexpression of amyloid beta in yeast cells. Table 1 lists names and National Center for Biotechnology Information (NCBI) Gene ID numbers for each of the yeast genes identified herein, non-limiting cellular function(s) of the encoded proteins, and results of screens performed on different media. One of skill in the art can readily obtain the NCBI RefSeq accession numbers corresponding to the nucleotide sequence (e.g., mRNA sequence) and protein sequences for each of the yeast genes and proteins identified herein. Asterisks in Table 1 indicate certain of these yeast genes that have identified human counterparts (discussed further below).

TABLE 1

| | | | Medium | | | |
|---|---|---|---|---|---|---|
| | | Gal | Glu | Gly | Gene ID | Function |
| Suppressors | WHI5 | Sup | Sup | Sup | 854249 | S repressing transcription factor |
| | SLF1** | | Sup | Sup | 852127 | RNA binding polysomes/copper resistance |
| | YBL086C | | Sup | Sup | 852192 | Unknown |
| | YAP1802* | Sup | Sup | Sup | 853157 | Clathrin cage assembly |
| | YPL014W | Sup | Sup | Sup | 856093 | Unknown |
| | RTS1* | Sup | Sup | | 854179 | Phosphatase subunit (of PP2A) |
| | SPT21 | Sup | Sup | Sup | 855217 | histone transcription factor/Ty suppressor |
| | FMP48** | Sup | Sup | Sup | 852943 | Unknown |
| | PPR1 | Sup | Sup | Sup | 850701 | Zinc-finger Uracil transcription factor |
| | TEC1** | Sup | Sup | Sup | 852377 | TY expression transcription factor |
| | ADE12* | Sup | Sup | Sup | 855501 | Adenylosuccinate synthase |
| | CRM1* | Sup | Sup | Sup | 853133 | Nucleus export |
| | NAB3 | | Sup | Sup | 855911 | Splicing/Non-poly A termination |
| | SLA1*** | | Sup | Sup | 852276 | Cortical actin assembly |
| | RTG3** | | Sup | Sup | 852171 | Retrograde/TOR transcription factor |
| | SRO9** | Sup | Sup | Sup | 850320 | RNA binding/actin organization |
| | MBP1** | Sup | Sup | Sup | 851503 | Transcription factor G1-S phase |
| | MUM2 | Sup | Sup | Sup | 852348 | Meiotic DNA replication |
| | INP52* | Sup | Sup | Sup | 855618 | Phosphatase/role in endocytosis |
| | FCY21 | | | Sup | 856788 | Purine-cytosine permease |
| | GRR1* | Enh | Sup | Sup | 853552 | Ubiquitin-ligation |
| | VPS9* | | Sup | Sup | 854876 | Vesicle mediated vacuolar protein transport |
| | OPY1 | Sup | Sup | Sup | 852426 | Unknown/OE blocks pheromone G1 arrest |
| Enhancers | SPO7 | Enh | Enh | Enh | 851224 | Phosphatase subunit/premeiotic DNA replication |
| | KAR9 | Enh | Enh | Enh | 855859 | Spindle position/microtuble orientation |
| | POG1 | Enh | Enh | Enh | 854684 | G1 transcription factor/inhibts alpha arrest |
| | KEM1* | Enh | Enh | Enh | 852702 | Exonuclease/P-bodies (Kar enhancing mutation) |
| | ROM1** | Enh | Enh | Enh | 852961 | GDP/GTP exchange for Rho1 |
| | MID2 | Enh | Enh | Enh | 851042 | Senses cell wall integrity |
| | BOP3 | Enh | Enh | Enh | 855686 | Unknown (Bypass of Pam1, which suppresses loss of PP2A) |
| | PMT2* | Enh | Enh | Enh | 851210 | Protein O-MannosylTransferase |
| | PSK1** | Enh | Enh | Enh | 851216 | S/T kinase |
| | YBL061C | Enh | Enh | Enh | 852218 | Activator of chitin synthase |
| | (SKT5)** | | | | | |
| | PET111 | Enh | | Enh | 855299 | Mitochondrial translation activator |
| | SLS1 | Enh | Enh | Enh | 850830 | Coordinates expression in mitochondria |
| | SVL3 | Enh | Enh | Enh | 856075 | Unknown/potential vacuolar function |
| | IVY1 | | Enh | Enh | 851815 | Phospholioid binding/trafficking |
| | MVP1* | Enh | Enh | | 855016 | Vacuole sorting |
| | PBS2* | Enh | | Enh | 853313 | Kinase/Osmotic stress/Ty regulator |
| | PKC1** | Enh | Enh | | 852169 | Kinase/cell wall remodeling |

It is expected that modulating expression of the genes and/or activity of proteins encoded by the genes will result in modulation of amyloid beta mediated toxicity, e.g., in amyloid beta expressing cells. For example, for those genes that were found to suppress toxicity when overexpressed in yeast, it is expected, in some embodiments, that enhancing expression of the genes and/or activity of proteins encoded by the genes will result in a suppression of amyloid beta toxicity, e.g., in amyloid beta expressing cells. Conversely, for those genes that were found to enhance toxicity when overexpressed in yeast, it is expected, in some embodiments, that inhibiting expression of the genes and/or the activity of proteins encoded by the genes will result in a suppression of amyloid beta toxicity, e.g., in amyloid beta expressing cells. For those genes that were found to suppress toxicity when overexpressed in yeast, it is expected, in some embodiments, that inhibiting expression of the genes and/or activity of proteins encoded by the genes will result in enhancement of amyloid beta toxicity, e.g., in amyloid beta expressing cells.

It is expected that the mechanisms by which amyloid beta induces toxicity in the yeast model system described herein is similar to the mechanisms by which amyloid beta induces toxicity in human cells. It will be appreciated that such toxicity can be manifested to a variety of extents and in a variety of ways ranging from dysfunction to death. A number of the yeast genes identified as modulating amyloid beta mediated toxicity in yeast cells have orthologous or highly related genes in humans. As a result, human counterparts of the identified yeast genes (and their encoded proteins) are expected to be useful targets for modulating, e.g., suppressing, amyloid beta mediated toxicity in human cells, and are of particular interest. In general, “counterparts” of yeast genes, e.g., human counterparts, may be identified based on sequence similarity, structural similarity, and/or functional similarity. In one embodiment, Homologene (a system for automated detection of homologs among the annotated genes of various completely sequenced eukaryotic genomes, available at www.ncbi.nlm.nih.gov/homologene) is used. Of course other means of identifying homologs based on sequence could be used. In some embodiments of any aspect of the invention relating to a human gene, the human gene is a homolog of a yeast suppressor or enhancer identified herein, wherein the homology can be identified in Homologene or based on homologous function.

Counterparts of at least some of the identified yeast genes and encoded proteins are present in a variety of nonhuman multicellular organisms, e.g., worms, flies, mice, rats, and/or non-human primates, and the identification of such non-human counterparts is an aspect of the invention. Such genes, and their encoded gene products, may be used in various aspects of the invention. For example, such genes can be overexpressed, e.g., in cells of their organism of origin, and used to modulate amyloid beta-mediated toxicity in such cells and/or to facilitate screening for, producing, or characterizing compounds that modulate amyloid beta-mediated toxicity.

Genes identified with single asterisks in Tables 1 (and 2) have identified human homologs based on Homologene and BLAST searching, or both. Genes identified with two asterisks have identified human homologs based at least on BLAST searching. Genes identified with three asterisks have human homologs based at least on functional similarity. Table 2 lists National Center for Biotechnology Information (NCBI) Gene ID numbers for certain of the human genes identified herein. As detailed in the following sections, the nucleotide and protein sequences of these genes and their encoded protein can be used to generate and/or identify compounds (including but not limited to nucleic acids, peptides, antibodies, small molecules) that modulate expression of genes or activity of encoded gene products. One of skill in the art will appreciate that a number of the genes identified herein encode more than one isoform of a particular protein (e.g., splice variants or forms that use alternative start codons). The invention encompasses embodiments directed to each such isoform, e.g., each such isoform having a Reference Sequence (RefSeq) in the NCBI RefSeq database. In some embodiments, an aspect of the invention relates to an isoform is normally expressed in the human brain (e.g., in at least part of one or more brain regions that is typically affected in individuals with AD, such as the hippocampus and/or cerebral cortex). In some embodiments, an aspect of the invention relates to an isoform that is normally expressed in healthy adults, e.g., in the brain. In some embodiments, expression of an isoform is altered in individuals with an amyloid beta mediated disorder, e.g., AD, as compared with individuals not having the disorder.

TABLE 2
Yeast suppressors and enhancers with identified human homologs

| | Yeast Gene | Human Homolog | Human Gene ID |
|---|---|---|---|
| Suppressors | SLF1** | LARP1 | 23367 |
| | YAP1802* | PICALM | 8301 |
| | RTS1* | PPP2R5C | 5527 |
| | FMP48** | MARK4 | 57787 |
| | TEC1** | TEAD2 | 8463 |
| | ADE12* | ADSSL1 | 122622 |
| | CRM1* | XPO1 | 7514 |
| | SLA1*** | SH3KBP1 | 30011 |
| | RTG3** | MITF | 4286 |
| | SRO9** | LARP1B | 55132 |
| | MBP1** | DAPK1 | 1612 |
| | INP52* | SYNJ1 | 8867 |
| | GRR1* | FBXL2 | 25827 |
| | VPS9* | RABGEF1 | 27342 |
| Enhancers | KEM1* | XRN1 | 54464 |
| | ROM1** | NET1 | 10276 |
| | PMT2* | POMT2 | 29954 |
| | PSK1** | PASK | 23178 |
| | YBL061C | SEL1L2 | 80343 |
| | (SKT5)** | | |
| | MVP1* | SNX8 | 29886 |
| | PBS2* | MAP2K4 | 6416 |
| | PKC1** | PKN2 | 5586 |

A summary of certain yeast Aβ suppressors and enhancers of particular interest and their human homologs (and non-limiting cellular function(s) thereof) is presented in Table 3.

TABLE 3
Selected yeast suppressors and enhancers and human homologs thereof

| | | Human |
|---|---|---|
| | Cellular Function | Homolog |
| Yeast Aβ | | |
| Suppressors | | |
| YAP1802 | Endocytosis | PICALM |
| INP52 | Endocytosis | SYNJ1 |
| SLA1 | Endocytosis | SH3KBP1 |
| RTS1 | Phosphatase regulation | PPP2R5C |
| ADE12 | Adenylosuccinate synthesis | ADSSL1 |
| CRM1 | Nuclear protein export | XPO1 |
| GRR1 | Ubiquitination | FBXL2 |
| VPS9 | Vesicle transport | RABGEF1 |
| Yeast Aβ | | |
| Enhancers | | |
| PBS2 | Osmotic stress response | MAP2K4 |
| KEM1 | RNA processing | XRN1 |
| MVP1 | Vacuolar sorting | SNX8 |
| PMT2 | Mannosylation | POMT2 |

The genes identified herein as modulators of amyloid beta mediated toxicity are sometimes referred to in subsequent sections (e.g., regarding screening assays) as “target genes” and the encoded proteins are sometimes referred to as “target proteins.” Names of genes provided herein should be understood to encompass reference to the gene and its encoded protein(s) unless otherwise indicated or otherwise evident from the context. For example, where referring to modulation (e.g., inhibition or enhancement) of expression of a gene, such reference should be understood to encompass modulating expression of the gene at the level of mRNA and/or protein in various embodiments (e.g., modulating the level of mRNA and/or modulating the level of protein (which may also be referred to as modulating expression of the protein). Modulation of activity as used herein typically refers to modulating activity of a product encoded by a gene, which product is typically a protein.

In accordance with certain aspects of the invention, compounds that modulate (e.g., enhance or suppress) the expression or activity of SPO7, KAR9, POG1, KEM1 (or human XRN1), ROM1 (or human NET1), MID2, BOP3, PMT2 (or human POMT2), PSK1 (or human PASK), YBL061C (or human SEL1L2), PET111, SLS1, SVL3, IVY1, MVP1 (or human SNX8), PBS2, (or human MAP2K4), PKC1 (or human PKN2), WI-115, SLF1 (or human LARP1), YBL086C, YAP1802 (or human PICALM), YPL014W, RTS1 (or human PPP2R5C), SPT21, FMP48 (or human MARK4), PPR1, TEC1 (or human TEAD2), ADE12 (or human ADSSL1), CRM1 (or human XPO1), NAB3, SLA1 (or human SH3KBP1), RTG3 (or human MITF), SRO9 (or human LARP1B), MBP1 (or human DAPK1), MUM2, INP52 (or human SYNJ1), FCY21, GRR1 (or human FBXL2), VPS9 (or human RABGEF1), or OPY1, modulate amyloid beta-mediated toxicity.

In general it is expected that enhancing the expression or activity of yeast suppressors or their human counterparts would suppress amyloid beta-mediated toxicity. However, in some embodiments of the invention, inhibiting the expression or activity of certain of these gene(s) may suppress amyloid beta-mediated toxicity.

In some embodiments, compounds that enhance the expression or activity of WHI5, SLF1 (or human LARP1), YBL086C, YAP1802 (or human PICALM), YPL014W, RTS1 (or human PPP2R5C), SPT21, FMP48 (or human MARK4), PPR1, TEC1 (or human TEAD2), ADE12 (or human ADSSL1), CRM1 (or human XPO1), NAB3, SLA1 (or human SH3KBP1), RTG3 (or human MITF), SRO9 (or human LARP1B), MBP1 (or human DAPK1), MUM2, INP52 (or human SYNJ1), FCY21, GRR1 (or human FBXL2), VPS9 (or human RABGEF1), or OPY1 protein are, in general, expected to suppress amyloid beta-mediated cellular toxicity. It is also understood that, in some embodiments, compounds capable of inhibiting the expression or activity of an inhibitor of WHI5, SLF1 (or human LARP1), YBL086C, YAP1802 (or human PICALM), YPL014W, RTS1 (or human PPP2R5C), SPT21, FMP48 (or human MARK4), PPR1, TEC1 (or human TEAD2), ADE12 (or human ADSSL1), CRM1 (or human XPO1), NAB3, SLA1 (or human SH3KBP1), RTG3 (or human MITF), SRO9 (or human LARP1B), MBP1 (or human DAPK1), MUM2, INP52 (or human SYNJ1), FCY21, GRR1 (or human FBXL2), VPS9 (or human RABGEF1), or OPY1 are, in general, expected to suppress amyloid beta-mediated cellular toxicity.

In some embodiments, compounds that inhibit the expression or activity of SPO7, KAR9, POG1, KEM1 (or human XRN1), ROM1 (or human NET1), MID2, BOP3, PMT2 (or human POMT2), PSK1 (or human PASK), YBL061C (or human SEL1L2), PET111, SLS1, SVL3, IVY1, MVP1 (or human SNX8), PBS2, (or human MAP2K4), or PKC1 (or human PKN2) (or that inhibit the activity of a protein encoded by any of the foregoing) are expected to inhibit amyloid beta-mediated cellular toxicity. It is also understood that, in some embodiments of the invention, compounds capable of enhancing the expression or activity of an inhibitor of SPO7, KAR9, POG1, KEM1 (or human XRN1), ROM1 (or human NET1), MID2, BOP3, PMT2 (or human POMT2), PSK1 (or human PASK), YBL061C (or human SEL1L2), PET111, SLS1, SVL3, IVY1, MVP1 (or human SNX8), PBS2, (or human MAP2K4), or PKC1 (or human PKN2) are expected to inhibit amyloid beta-mediated cellular toxicity.

Genes (and their encoded proteins) that act in the same biological pathway or process as an identified yeast gene or human counterpart (or protein encoded by such yeast gene or human counterpart) may also be useful targets for modulating, e.g., suppressing, amyloid beta mediated toxicity in human cells. One aspect of the invention is the identification of certain biological processes and pathways that involve one or more genes identified herein as modulators of amyloid beta induced toxicity. Proteins (and the genes that encode them) that serve as part of the same multimolecular complex as a protein encoded by an identified yeast gene or human counterpart may also be useful targets for modulating, e.g., suppressing, amyloid beta mediated toxicity, e.g., in human cells. For example, if a protein serves as a subunit of a multi-subunit protein, other subunit(s) of the same protein (and the genes that encode them) and/or the protein as a whole may be useful targets for modulating, e.g., suppressing, amyloid beta mediated toxicity in human cells. A number of the identified yeast genes and their human counterparts encode polypeptides that serve as subunits of multi-subunit proteins. For example, yeast RTS1 was identified as a suppressor of amyloid beta toxicity. RTS1 and its human counterpart (PPP2R5C) are regulatory subunits of yeast and human protein phosphatase 2A (PP2A), respectively. Other subunits of PP2A may also be useful targets for modulating, e.g., suppressing, amyloid beta mediated toxicity.

Notably, three of the twelve genes with human homologs (as identified using Homologene) have functions related to clathrin-mediated endocytosis (YAP1802, SLA1, and INP52), while a fourth, VPS9, functions in vesicle transport. (The human homologs are PICALM, SH3KBP1, SYNJ1, and RABGEF1, respectively). Thus the instant invention identifies PICALM, SH3KBP1, SYNJ1, RABGEF1, and other human genes involved in endocytosis and/or protein trafficking, and regulators of the expression of these genes or the activity of their encoded proteins, as suitable targets for compounds that promote detoxification of Aβ or otherwise inhibit Aβ-mediated toxicity.

In some embodiments of any aspect of the invention, the gene (or set of genes) is a human gene (or set of genes, such as the set of homologs of yeast enhancers or the set of homologs of yeast suppressors) selected from those listed in Table 3 and/or the protein (or set of proteins such as the set of homologs of yeast enhancers or the set of homologs of yeast suppressors) is a human protein (or set of proteins) selected from those listed in Table 3.

In some embodiments of any aspect of the invention, the gene (or set of genes such as the set of homologs of yeast enhancers or the set of homologs of yeast suppressors) is a human gene (or set of genes such as the set of homologs of yeast enhancers or the set of homologs of yeast suppressors) selected from those listed in Table 2 and notated with two asterisks and/or the protein (or set of proteins such as the set of homologs of yeast enhancers or the set of homologs of yeast suppressors) is a human protein (or set of proteins such as the set of homologs of yeast enhancers or the set of homologs of yeast suppressors) selected from those listed in Table 2 and notated with two asterisks.

### Screening Assays

Certain aspects of the present disclosure provide methods of screening for a candidate drug (agent or compound) or a genetic factor that modulates amyloid beta-induced toxicity. Various types of candidate drugs may be screened by the methods described herein, including nucleic acids, polypeptides, small molecule compounds, and peptidomimetics. In some cases, genetic agents can be screened by contacting the yeast cell with a nucleic acid construct coding for a gene. For example, one may screen cDNA libraries expressing a variety of genes, to identify genes that modulate amyloid beta-induced toxicity.

For example, the identified drugs may modulate amyloid beta-induced toxicity. Accordingly, irrespective of the exact mechanism of action, drugs identified by the screening methods described herein are expected to provide therapeutic benefit to Alzheimer's disease.

In certain embodiments, screening methods described herein use yeast cells that are engineered to express an amyloid beta protein. For chemical screens, suitable mutations of yeast strains designed to affect membrane efflux pumps and increase permeability for drugs can be used. For example, a yeast strain bearing mutations in the ERG6 gene, the PDR1 gene, the PDR3 gene, and/or the PDR5 gene is contemplated of use. For example, a yeast strain bearing mutations in membrane efflux pumps (erg6, pdr1, pdr3, and/or pdr5) has been successfully used in many screens to identify growth regulators (Jensen-Pergakes K L, et al., 1998. Antimicrob Agents Chemother 42:1160-7).

Methods of the present disclosure relate to identifying compounds or genes tha modulate amyloid beta-induced toxicity. One of the strongest aspects of yeast is the possibility of performing high throughput screens that may identify genes, peptides and other compounds with the potential to ameliorate toxicity. A large number of compounds can be screened under a variety of growth conditions and in a variety of genetic backgrounds. The toxicity screen has the advantage of not only selecting for compounds that interact with amyloid beta, but also upstream or downstream targets that are not themselves cytotoxic and that are not yet identified.

In certain embodiments, candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd.(Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can be used in the screens.

Another embodiment relates to genetic screens. For example, genomic libraries and disruption libraries can be screened to find extragenic suppressors or enhancers of amyloid beta-induced toxicity. Because the yeast genome is small, 10,000 transformants of each type should be sufficient for good coverage.

Another embodiment contemplates screening assays using fluorescent resonance energy transfer (FRET). FRET occurs when a donor fluorophore is in close proximity (10-60 A) to an acceptor fluorophore, and when the emission wavelength of the first overlaps the excitation wavelength of the second (Kenworthy A K et al., 2001. Methods. 24:289-96). FRET should occur when cyan fluorescent protein (CFP) and yellow fluorescent protein (YFP) fusion proteins are actually part of the same complex.

For example, an amyloid beta protein can be fused to CFP and to YFP respectively, and integrated in the yeast genome under the regulation of a GAL1-10 promoter. Cells are grown in galactose to induce expression. Upon induction, cells produce the fusion proteins, which aggregate and bring the CFP and YFP close together. Because proteins in the aggregates are tightly packed, the distance between the CFP and YFP is less than the critical value of 100 A that is necessary for FRET to occur. In this case, the energy released by the emission of CFP will excite the YFP, which in turn will emit at its characteristic wavelength. FRET based screening can be used to identify candidate compounds including, drugs, genes or other factors that can disrupt the interaction of CFP and YFP by maintaining the proteins in a state that does not allow aggregation to occur.

One embodiment contemplates screening assays using fluorescence activated cell sorting (FACS) analysis. FACS provides the means of scanning individual cells for the presence offluorescently labeled/tagged moiety. The method is unique in its ability to provide a rapid, reliable, quantitative, and multiparameter analysis on either living or fixed cells. For example, an amyloid beta protein can be suitably labeled, and provide a useful tool for the analysis and quantitation of protein aggregation as a result of other genetic or growth conditions of individual yeast cells as described above.

Screens (e.g., for compounds and/or for genetic suppressors or enhancers) can be carried out under a variety of different conditions. For example, a variety of different culture media can be used. Culture media can contain different carbon sources, e.g., different sugars such as glucose, glycerol, galactose, raffinose, etc. In some embodiments, multiple screens are performed using two, three, or more different culture conditions (e.g., culture media containing different carbon sources), and compounds or genes identified as “hits” under at least two different culture conditions are identified. In some embodiments, screens are performed under two or more different culture conditions (e.g., using culture media containing different carbon sources), wherein the different culture conditions (e.g., different carbon sources) result in different levels of mitochondrial respiration. For example, growth using culture media containing glucose, glycerol, or galactose result in different levels of mitochondrial respiration. In glucose, yeast cells ferment and respiration remains low until all glucose is converted to ethanol. In galactose respiration is moderately active. In glycerol, yeast cells are completely dependent on respiration for growth. In some embodiments, a screen is performed in parallel using media containing glucose, galactose, or glycerol as a carbon source.

Certain embodiments provide methods of further testing those potential drugs that have been identified in the yeast system, in other model systems. The model systems include, but are not limited to, worms, flies, mammalian cells, and in vivo animal models. Certain embodiments provide methods of testing genes and proteins identified herein (and counterparts thereof) in other model systems. For example, worm, fly, rodent (e.g., mouse or rat) counterparts of yeast genes identified herein can be overexpressed or inhibited in worms, flies, rodent(s), respectively, or in cells derived from such organisms (e.g., primary cells or cell lines, e.g., immortalized cell lines). The invention provides such non-human organisms and cells. Optionally, the cells are isolated cells. In some embodiments, the cells are isolated from an organism that expresses the gene. In some embodiments, an existing cell line is genetically engineered to express or lack expression of the gene. Human genes identified herein can be overexpressed or inhibited in human cells, e.g., isolated cells, which may be primary cells or a cell line. The invention provides such human cells. In one aspect, the invention provides a method comprising overexpressing or inhibiting a human gene identified herein in human cells that are susceptible to amyloid beta mediated toxicity and determining the effect of such overexpression or inhibition on such toxicity. For example, the cells can be neurons exposed to amyloid beta aggregates, e.g., amyloid beta oligomers and/or dimers. Aβ oligomers and dimers have been proposed to be the toxic species in AD (Li et al., (2009) Neuron 62, 788; Kayed et al. (2003) Science 300, 486.

The methods described herein further include methods (also referred to herein as “target gene screening assays”) for identifying compounds that modulate (i.e., increase or decrease) expression or activity of selected target genes or their protein products. Such compounds include, e.g., polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to the target proteins, or have a stimulatory or inhibitory effect on, for example, expression of a target gene or activity of a target protein. Compounds thus identified can be used to modulate the expression or activity of target genes or target proteins in a therapeutic protocol and/or in further screening methods.

In some embodiments, target gene screening assays of the invention involve assaying the effect of a test compound on expression or activity of a target nucleic acid or target protein in a test sample (i.e., a sample containing the target nucleic acid or target protein). Expression or activity in the presence of the test compound can be compared to expression or activity in a control sample (i.e., a sample containing the target protein that is incubated under the same conditions, but without the test compound). A change in the expression or activity of the target nucleic acid or target protein in the test sample compared to the control indicates that the test compound modulates expression or activity of the target nucleic acid or target protein and is a candidate agent. Compounds can be tested for their ability to modulate one or more activities mediated by a target protein described herein. For example, compounds that modulate expression of a gene or activity of a protein listed in Table 1, 2, and/or 3 can be tested for their ability to modulate toxicity in cells expressing amyloid beta. Methods of assaying a compound for such activities are known in the art. In some cases, a compound is tested for its ability to directly affect target gene expression or binding to a target protein (e.g., by decreasing the amount of target RNA in a cell or decreasing the amount of target protein in a cell) and/or tested for its ability to modulate a metabolic effect or phenotype associated with the target protein.

In one embodiment, assays are provided for screening candidate or test molecules that are substrates of a target protein or a biologically active portion thereof in a cell. In another embodiment, the assays are for screening candidate or test compounds that bind to a target protein or modulate the activity of a target protein or a biologically active portion thereof. Such compounds include those that disrupt the interaction between a target protein and its ligand or receptor.

In one embodiment, a cell-based assay is employed in which a cell that expresses a target protein or biologically active portion thereof is contacted with a test compound. The ability of the test compound to modulate expression or activity of the target protein is then determined. The cell, for example, can be a yeast cell or a cell of mammalian origin, e.g., rat, mouse, or human.

The ability of the test compound to bind to a target protein or modulate target protein binding to a compound, e.g., a target protein substrate, can also be evaluated. This can be accomplished, for example, by coupling the compound, e.g., the substrate, with a radioisotope or enzymatic label such that binding of the compound, e.g., the substrate, to the target protein can be determined by detecting the labeled compound, e.g., substrate, in a complex. Alternatively, the target protein can be coupled with a radioisotope or enzymatic label to monitor the ability of a test compound to modulate target protein binding to a target protein substrate in a complex. For example, compounds (e.g., target protein substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a target protein substrate) to interact with target protein with or without the labeling of any of the interactants can be evaluated. For example, a microphysiometer can be used to detect the interaction of a compound with a target protein without the labeling of either the compound or the target protein (McConnell et al., Science 257:1906-1912, 1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between a compound and a target protein.

In yet another embodiment, a cell-free assay is provided in which a target protein or biologically active portion thereof is contacted with a test compound and the ability of the test compound to bind to the target protein or biologically active portion thereof is evaluated. In general, biologically active portions of target proteins to be used in assays described herein include fragments that participate in interactions with other molecules, e.g., fragments with high surface probability scores.

Cell-free assays involve preparing a reaction mixture of the target protein and the test compound under conditions and for a time sufficient to allow the two components to interact and bind, thus forming a complex that can be removed and/or detected. The interaction between two molecules can also be detected using fluorescence energy transfer (FET) (see, for example, Lakowicz et al., U.S. Pat. No. 5,631,169; Stavrianopoulos et al, U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, the “donor” protein molecule may use the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

In another embodiment, the ability of a target protein to bind to a target molecule can be determined using real-time Biomolecular Interaction Analysis (BIA) (e.g., Sjolander et al., Anal. Chem., 63:2338-2345, 1991, and Szabo et al., Curr. Opin. Struct. Biol., 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In various of these assays, the target protein or the test substance is anchored onto a solid phase. The target protein/test compound complexes anchored on the solid phase can be detected at the end of the reaction. Generally, the target protein is anchored onto a solid surface, and the test compound (which is not anchored) can be labeled, either directly or indirectly, with detectable labels discussed herein.

It may be desirable to immobilize either the target protein, an anti-target protein antibody, or its target molecule to facilitate separation of complexed from uncomplexed forms of one or both of the proteins, as well as to accommodate automation of the assay. Binding of a test compound to a target protein, or interaction of a target protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S— transferase/target protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein. The mixture is then incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of target protein binding or activity determined using standard techniques. Other techniques for immobilizing a target protein on matrices include using conjugation of biotin and streptavidin. Biotinylated target protein can be prepared from biotin-NHS (N-hydroxy-succinimide) using techniques known in the art (e.g., biotinylation kit, Pierce Chemicals, Rockford, 111.), and immobilized in the wells of streptavidin-coated 96 well plates (Pierce Chemical). To conduct the assay, the non-immobilized component is added to the coated surface containing the anchored component. After the reaction is complete, unreacted components are removed (e.g., by washing) under conditions such that any complexes formed will remain immobilized on the solid surface. The complexes anchored on the solid surface can be detected in a number of ways. Where the previously non-immobilized component is pre-labeled, the presence of a label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In some cases, the assay is performed utilizing antibodies reactive with target protein, but which do not interfere with binding of the target protein to its target molecule. Such antibodies can be derivatized to the wells of the plate, and unbound target protein trapped in the wells by antibody conjugation. Methods for detecting such complexes, in addition to those described above for the GST-immobilized complexes, include immunodetection of complexes using antibodies reactive with the target protein or target molecule, as well as enzyme-linked assays which rely on detecting an enzymatic activity associated with the target protein.

Alternatively, cell-free assays can be conducted in a liquid phase. In such an assay, the reaction products are separated from unreacted components, by any of a number of standard techniques, including but not limited to: differential centrifugation (see, for example, Rivas and Minton, Trends Biochem. Sci., 18:284-7, 1993); chromatography (gel filtration chromatography, ion-exchange chromatography); electrophoresis (e.g., Ausubel et al., eds. Current Protocols in Molecular Biology 1999, J. Wiley: New York.); and immunoprecipitation (see, for example, Ausubel et al., eds., 1999, Current Protocols in Molecular Biology, J. Wiley: New York). Such resins and chromatographic techniques are known to one skilled in the art (e.g., Heegaard, J. MoI. Recognit, 11: 141-148, 1998; Hage et al., J. Chromatogr. B. Biomed. Sci. Appl., 699:499-525, 1997). Further, fluorescence energy transfer may also be conveniently utilized, as described herein, to detect binding without further purification of the complex from solution.

The assay can include contacting the target protein or a biologically active portion thereof with a known compound that binds to the target protein to form an assay mixture, contacting the assay mixture with a test compound, and determining the ability of the test compound to interact with the target protein, wherein determining the ability of the test compound to interact with the target protein includes determining the ability of the test compound to preferentially bind to the target protein or biologically active portion thereof, or to modulate the activity of a target molecule, as compared to the known compound.

A target protein can, in vivo, interact with one or more cellular or extracellular macromolecules, such as proteins. For the purposes of this discussion, such cellular and extracellular macromolecules are referred to herein as “binding partners.” Compounds that disrupt such interactions are useful for regulating the activity of the target protein. Such compounds can include, but are not limited, to molecules such as antibodies, peptides, and small molecules. In general, target proteins for use in identifying agents that disrupt interactions are the target proteins identified herein. In alternative embodiments, the invention provides methods for determining the ability of the test compound to modulate the activity of a target protein through modulation of the activity of a downstream effector of a target protein. For example, the activity of the effector molecule on an appropriate target can be determined, or the binding of the effector to an appropriate target can be determined, as described herein. To identify compounds that interfere with the interaction between the target protein and its binding partner(s), a reaction mixture containing the target protein and the binding partner is prepared, under conditions and for a time sufficient, to allow the two products to form a complex. To test an inhibitory agent, the reaction mixture is provided in the presence (test sample) and absence (control sample) of the test compound. The test compound can be initially included in the reaction mixture, or can be added at a time subsequent to the addition of the target gene and its cellular or extracellular binding partner. Control reaction mixtures are incubated without the test compound or with a control compound. The formation of complexes between the target protein and the cellular or extracellular binding partner is then detected. The formation of a complex in the control reaction, and less formation of complex in the reaction mixture containing the test compound, indicates that the compound interferes with the interaction of the target protein and the interactive binding partner. Such compounds are candidate compounds for inhibiting the expression or activity or a target protein. Additionally, complex formation within reaction mixtures containing the test compound and normal target protein can also be compared to complex formation within reaction mixtures containing the test compound and mutant target gene product. This comparison can be important in those cases wherein it is desirable to identify compounds that disrupt interactions of mutant but not normal target protein.

Binding assays can be carried out in a liquid phase or in heterogenous formats. In one type of heterogeneous assay system, either the target protein or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface. To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or mat disrupt preformed complexes can be detected.

In another embodiment, modulators of target expression (RNA or protein) are identified. For example, a cell or cell-free mixture is contacted with a test compound and the expression of target mRNA or protein evaluated relative to the level of expression of target mRNA or protein in the absence of the test compound. When expression of target mRNA or protein is greater in the presence of the test compound than in its absence, the test compound is identified as a stimulator (candidate compound) of target mRNA or protein expression. Alternatively, when expression of target mRNA or protein is less (statistically significantly less) in the presence of the test compound than in its absence, the test compound is identified as an inhibitor (candidate compound) of target mRNA or protein expression. The level of target mRNA or protein expression can be determined by methods described herein and methods known in the art such as Northern blot, microarray hybridization, or reverse transcription (RT-PCR) for detecting target mRNA or Western blot for detecting target protein, respectively.

In another aspect, the methods described herein pertain to a combination of two or more of the assays described herein. For example, a modulating agent can be identified using a cell-based or a cell-free assay, and the ability of the agent to modulate the activity of a target protein can be confirmed in vivo, e.g., in an animal such as an animal model for Alzheimer's disease. This invention further pertains to novel agents identified by the above-described screening assays. Accordingly, it is within the scope of this invention to further use an agent (compound) identified as described herein (e.g., a target protein modulating agent, an anti sense nucleic acid molecule, an siRNA, a target protein-specific antibody, or a target protein-binding partner) in an appropriate animal model to determine the efficacy, toxicity, side effects, or mechanism of action, of treatment with such an agent. Furthermore, novel agents identified by the above-described screening assays can be used for treatments as described herein. Compounds that modulate target protein expression or activity (target protein modulators) can be tested for their ability to affect biological or biochemical effects associated with the target protein, e.g., with decreased expression or activity of target protein using methods known in the art and methods described herein. For example, the ability of a compound to modulate amyloid beta mediated toxicity can be tested using an in vitro or in vivo model for Alzheimer's disease.

### Modulators of Target Gene or Protein Expression or Activity

Methods of modulating target gene or protein expression or activity can be accomplished using a variety of compounds including nucleic acid molecules that are targeted to a target nucleic acid sequence or fragment thereof, or to a target protein. Compounds that may be useful for inhibiting or enhancing target protein expression or activity include polynucleotides, polypeptides, small non-nucleic acid organic molecules, small inorganic molecules, antibodies or fragments thereof, antisense oligonucleotides, siRNAs, and ribozymes. Exemplary methods of identifying and/or producing such compounds are described herein.

### Nucleic Acids

Molecules, e.g., nucleic acids, that are targeted to a target RNA are useful for certain of the methods described herein, e.g., inhibition of target protein expression, e.g., for treating an amyloid beta mediated disease such as Alzheimer's disease. Examples of such nucleic acids include double-stranded RNA such as short interfering RNAs (siRNAs) that inhibit gene expression by RNA interference (RNAi). Nucleic acid molecules or constructs that are useful as described herein include siRNA molecules that comprise, e.g., 15-30 nucleotides in each strand, e.g., 15, 16, 17, 18, 19, 20, 21, 22, 23, 24, 25, 26, 27, 28, 29, or 30 nucleotides in each strand (e.g., 19-21 nucleotides), wherein one of the strands is substantially complementary, e.g., at least 80% (or more, e.g., 85%, 90%, 95%, or 100%) complementary, e.g., having 3, 2, 1, or 0 mismatched (i.e., non-complementary) nucleotide(s), to a target region in a target mRNA, and the other strand is substantially complementary to the first strand. In some embodiments, the siRNA comprises a double-stranded portion at least 15 nucleotides in length, e.g., between 15-30 nucleotides in length. In some embodiments, the siRNA comprises a 3′ overhang on one or both strands. In some embodiments, the 3′ overhang is between 1 and 5 nucleotides, e.g., 1, 2, 3, 4, or 5 nucleotides in length, wherein the lengths can be the same or different on the two strands.

dsRNA molecules (e.g., siRNA) can be produced using methods known in the art. For example, they can be chemically synthesized, can transcribed be in vitro or in vivo from a DNA template. The dsRNA molecules can be designed using methods known in the art. A variety of different computer programs are available to assist in siRNA design. See, e.g., Muhonen P & Holthofer H (2010) Methods Mol. Biol. 2010; 623:93-107 for review and links to websites of numerous siRNA design tools. The sequence of an siRNA can be selected to reduce the likelihood of “off-target” effects. For example, a sequence unique in the genome (e.g., unique in the human genome) can be selected as a target sequence for siRNA design. Negative control siRNAs (“scrambled”) can be used, if desired, to confirm the effect of an siRNA on a target gene expression and/or to confirm the effect of the siRNA on amyloid beta mediated toxicity. Such siRNAs generally have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. Controls can also be designed by introducing an appropriate number of base mismatches into the selected siRNA sequence.

The nucleic acid compositions that are useful for certain of the methods described herein include both siRNA and crosslinked siRNA derivatives. Crosslinking can be used to alter the pharmacokinetics of the composition, for example, to increase half-life in the body. Thus, the invention includes siRNA derivatives that include siRNA having two substantially complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some cases, the siRNA or siRNA derivative has, e.g., at its 3′ terminus, a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA or siRNA derivatives in this way can improve cellular uptake or enhance cellular targeting activities of the resulting siRNA or siRNA derivative as compared to the corresponding unmodified siRNA or siRNA derivative, are useful for tracing the siRNA or siRNA derivative in the cell, or improve the stability of the siRNA derivative compared to the corresponding siRNA.

microRNA (miRNAs) are endogenous noncoding RNAs of approximately 22 nucleotides in length that can regulate gene expression at the post transcriptional or translational level. A large number of animal genes are regulated by miRNAs, sometimes in a cell or tissue-specific or developmental stage-specific manner. miRNAs are excised from an approximately 70 nucleotide precursor RNA stem-loop, which in turn is derived from a longer RNA precursor. By substituting the stem sequences of an miRNA precursor with sequence substantially complementary to the target mRNA, a vector construct that expresses the novel miRNA can be used to produce siRNAs to initiate RNAi against specific mRNA targets in mammalian cells. In other embodiments, a naturally occurring endogenous miRNA that inhibits expression of a target gene is identified or an artificial miRNA is designed. Such miRNA can then be overexpressed or delivered to cells or subjects (in order to inhibit the target gene) or their effects can be inhibited (e.g., using synthetic oligonucleotides termed “antagomirs” that are antisense to the miRNA) in order to upregulate expression of the target gene.

Antisense nucleic acids are useful for inhibiting expression of a target protein in certain embodiments of the invention. Antisense nucleic acid molecules are single-stranded molecules whose nucleotide sequence is substantially complementary to all or part of an RNA, e.g., a mRNA encoding a target protein. An antisense nucleic acid molecule can be antisense to all or part of a non-coding region or coding region of the coding strand of a nucleotide sequence encoding a target protein. As known in the art, non-coding regions (“5′ and 3′ untranslated regions”) are the 5′ and 3′ sequences that flank the coding region and are not translated into amino acids. Based upon the nucleotide sequences disclosed herein, one of skill in the art can select and synthesize any of a number of appropriate antisense molecules to target a gene identified herein. For example, a series of oligonucleotides of 15-30 nucleotides spanning the length of a nucleic acid (e.g., a target nucleic acid) can be prepared, followed by testing for inhibition of expression of the gene. An antisense oligonucleotide can be, for example, about 5, 10, 15, 20, 25, 30, 35, 40, 45, or 50 nucleotides or more in length. An antisense nucleic acid described herein can be constructed using chemical synthesis and/or enzymatic ligation reactions using procedures known in the art.

In some cases, a pool of siRNAs or miRNAs or antisense molecules is used to modulate the expression of a target gene. The pool is composed of at least 2, 3, 4, 5, 8, or different sequences targeted to the target gene.

Aptamers are nucleic acid molecules having a structure that permits them to specifically bind to protein ligands and offer a means by which target protein activity can be specifically decreased (see, e.g., Osborne, et al., Curr. Opin. Chem. Biol., 1: 5-9, 1997; and Patel, Curr. Opin. Chem. Biol., 1:32-46, 1997; Mayer G. (2009) Angew Chem Int Ed Engl. 48(15):2672-89). Aptamers can be identified using a technique termed systematic evolution of ligands by exponential enrichment (SELEX, reviewed in Stoltenburg R, et al. Biomol Eng. (2007) 24(4):381-403). The invention provides for identification of aptamers that specifically bind to and inhibit a target protein identified herein.

Ribozymes that have specificity for a target nucleic acid sequence can also be used to inhibit target gene expression. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. Methods of designing and producing ribozymes are known in the art (see, e.g., Scanlon, 1999, Therapeutic Applications of Ribozymes, Humana Press). A ribozyme having specificity for a target nucleic acid molecule or fragment thereof can be designed based upon the nucleotide sequence of a target cDNA. Alternatively, an mRNA encoding a target protein or fragment thereof can be used to select a catalytic RNA having a specific ribonuclease activity from a pool of RNA molecules (See, e.g., Bartel and Szostak, Science, 261:1411-1418, 1993).

Nucleic acid molecules that form triple helical structures can also be used to modulate target protein expression. For example, expression of a target protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene, Anticancer Drug Des., 6(6):569-84, 1991; Helene, Ann. N.Y. Acad. Sci., 660:27-36, 1992; and Maher, Bioassays, 14(12):807-15, 1992.

Nucleic acids (e.g., siRNAs, aptamers, or antisense oligonucleotides) can comprise standard nucleotides (A, G, C, T, U), non-standard nucleotides (which may or may not be naturally occurring nucleotides) or variously modified nucleotides designed, e.g., to increase the biological stability of the molecules or to increase the physical stability of the duplex formed between complementary nucleic acids. Nucleic acids can comprise nucleotides that comprise modified bases, modified backbones (e.g., modified sugars, and/or modified inter-nucleotide linkages (as compared with the bases, sugars, and phosphodiester backbone found in DNA and RNA). In some embodiments, the nucleic acid modification is selected to stabilize the nucleic acid (e.g., to reduce its sensitivity to nuclease(s)) or otherwise prolong its half-life in the body. Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, S-carboxymethylaminomethyl-Z-thioiiridiri-e, 5-carboxymethylaminomethyluracil,dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiour-acil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Modified sugars include, e.g., 2′-O alkyl derivatives. Modified inter-nucleotide linkages include, e.g., use of phosphorothioate derivatives, peptide nucleic acid, morpholino- and locked nucleic acid, as well as glycol nucleic acid and threose nucleic acid structures. See, e.g., Deleavey, G, et al., Current Protocols in Nucleic Acid Chemistry 16.3.1-16.3.22, 2009, for discussion of exemplary nucleic acid modifications of use in various embodiments of the invention, e.g., siRNA. Modification(s) may be present in one or both strands and may be located throughout the strand(s) or at particular positions in various embodiments. Multiple different modifications can be used in combination.

Exemplary routes of administration for nucleic acids (e.g., siRNAs, miRNAs, aptamers, or antisense nucleic acids) include direct administration, e.g., by injection, at a tissue site. For example, they may be administered directly to the brain. Alternatively, nucleic acid molecules, optionally modified to target selected cells or tissues, can be administered systemically. For example, for systemic administration, molecules can be modified such that they specifically bind to receptors or antigens expressed on a selected cell surface, e.g., by linking the nucleic acid molecules to peptides, small molecules, or antibodies that bind to cell surface receptors or antigens.

Nucleic acids, e.g., siRNAs, can be delivered into cells by a variety of means, e.g., cationic liposome transfection and electroporation. Sequences that are modified to improve their stability or cell uptake can be used. Such stabilized molecules are particularly useful for in vivo methods such as for administration to a subject to decrease target protein expression. Compounds, e.g., nucleic acids, e.g., siRNA, can be conjugated to a variety of moieties that target them to cells or tissues, e.g., the brain. For example, vitamin E (alpha tocopherol) can be conjugated to siRNAs, and the conjugated siRNAs optionally delivered with HDL. In some embodiments, a compound is conjugated to a moiety that binds to a receptor expressed by neurons. Expression can be achieved by delivering a vector (e.g., a viral vector) that causes the cell to express the siRNA molecule (or other nucleic acid) to a cell, e.g., a neuron. The vector can comprise a recombinant nucleic acid in which a sequence encoding a short hairpin RNA or miRNA precursor or sequence encoding an antisense RNA is operably linked to a promoter (e.g., a Pol II or Pol III promoter) that directs expression in a cell type of interest.

Viral-mediated delivery mechanisms include, e.g., recombinant adenoviruses, retroviruses, e.g., lentiviruses, and vectors comprising at least part of a genome derived from such virus(es) are also of use to express shRNA and/or miRNA precursors in cells, e.g., for research or therapeutic purposes.

Nanoparticles and liposomes can also be used to deliver nucleic acids, e.g., oligonucleotides, e.g., siRNAs, into cells or organisms (e.g., non-human animals or humans). Likewise, in some embodiments, viral gene delivery, direct injection, nanoparticle particle-mediated injection, or liposome injection may be used to express siRNA in cells or non-human animals or humans.

In some embodiments, cell-penetrating peptides (CPPs) or peptoids are used to facilitate cell uptake of a compound, e.g., a compound that modulates amyloid beta mediated toxicity and/or that modulates expression of a suppressor or enhancer of amyloid beta mediated toxicity. The compound may be, e.g., a nucleic acid such as a siRNA or aptamer. As known in the art, CPPs are peptides (typically arginine-rich) that are able to traverse cell membranes and transport a wide range of cargo molecules along with them (reviewed in Chugh A,. et al., (2010) IUBMB Life. 62(3):183-93). A CPP can be covalently or noncovalently attached to a compound to enhance uptake by cells (in vitro or in vivo).

In some embodiments, siRNAs or other compounds that inhibit target protein expression or activity are effective for ameliorating undesirable effects of amyloid beta when target RNA levels are reduced by at least 25%, 50%, 75%, 90%, or 95%. In some cases, it is desired that target RNA levels be reduced by not more than 10%, 25%, 50%, or 75%. Methods of determining the level of target gene expression (or reduction therein), are known in the art. For example, the level of target RNA can be determined, e.g., using Northern blot, microarray, RT-PCR, or RNA-SEQ on a sample from cell(s) (e.g., of a cell line) or a subject. Levels of target protein can also be measured using, e.g., Western blot, immunoassay method(s), etc. Such methods can be used, e.g., to assess the effect of a small molecule, peptide, antibody, nucleic acid, or other compound, on the target mRNA or protein.

### Polypeptides

Isolated target proteins, fragments thereof, and variants thereof are provided herein. These polypeptides can be used, e.g., as immunogens to raise antibodies, in screening methods, or in methods of treating subjects, e.g., by administration of the target proteins. An “isolated” or “purified” polypeptide or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of polypeptides in which the polypeptide of interest is separated from cellular components of the cells from which it is isolated or recombinantly produced. Thus, a polypeptide that is substantially free of cellular material includes preparations of polypeptides having less than about 30%, 20%, 10%, or 5% (by dry weight) of heterologous protein (also referred to herein as “contaminating protein”). In general, when the polypeptide or biologically active portion thereof is recombinantly produced, it is also substantially free of culture medium, i.e., culture medium represents less than about 20%, 10%, or 5% of the volume of the protein preparation. In general, when the polypeptide is produced by chemical synthesis, it is substantially free of chemical precursors or other chemicals, i.e., it is separated from chemical precursors or other chemicals that are involved in the synthesis of the polypeptide. Accordingly such preparations of the polypeptide have less than about 30%, 20%, 10%, or 5% (by dry weight) of chemical precursors or compounds other than the polypeptide of interest. Expression of target proteins can be assayed to determine the amount of expression. Methods for assaying protein expression are known in the art and include Western blot, immunoprecipitation, and radioimmunoassay.

As used herein, a “biologically active portion” of a target protein comprises a portion of a target protein that retains at least one biological function of a full length protein. The biologically active portion may have an activity that differs in magnitude from that of the full length protein. For example, the biologically active portion may have between 20% and 100% of the activity of the full length portion. In some embodiments, the biologically active portion may have increased activity relative to the full length portion (e.g., if the biologically active portion lacks an inhibitory domain present in the full length portion). In some embodiments, the biologically active portion may retain the ability to participate in an interaction with a second molecule, e.g., a different protein. Biologically active portions of a target protein include polypeptides that include fewer amino acids than a full-length target protein, and exhibit at least one activity of a target protein. In some embodiments, a biologically active portion includes a domain or motif with at least one activity of the target protein. In some embodiments, the activity is ability to suppress or enhance amyloid beta mediated toxicity. In some embodiments, the activity is an enzymatic activity.

A biologically active portion of a target protein can be a polypeptide that is, for example, 10, 25, 50, 100, 200 or more amino acids in length. It may be, for example, between 10% and 99.9% as long as the parent polypeptide. Biologically active portions of a target protein can be used as targets for developing agents that modulate a target protein mediated activity, e.g., compounds that inhibit target protein activity. In some embodiments, the target protein has a sequence identical to a sequence disclosed herein (e.g., an amino acid sequence found under an Accession Number listed in Table 1 or 2). Other useful polypeptides are substantially identical (e.g., at least about 45%, 55%, 65%, 75%, 85%, 95%, 96%, 97%, 98%, or 99% identical) to a sequence disclosed herein (e.g., an amino acid sequence found under an Accession Number listed in Table 1) and (a) retain at least one functional activity of the target protein yet differs in amino acid sequence due to natural allelic variation or mutagenesis, or (b) exhibits an altered functional activity (e.g., as a dominant negative) where desired. Provided herein are variants that have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the polypeptide. An antagonist of a polypeptide can inhibit one or more of the activities of the naturally occurring form of the polypeptide by, for example, competitively binding to a downstream or upstream member of a cellular signaling cascade that includes the polypeptide. Thus, specific biological effects can be elicited by treatment with a variant of limited function. Treatment of a subject with a variant having a subset of the biological activities of the naturally occurring form of the polypeptide can have fewer side effects in a subject relative to treatment with the naturally occurring form of the polypeptide. In some embodiments, the variant target protein is a dominant negative form of the target protein. Dominant negatives can be used, e.g., in methods in which inhibition of target protein action is desired. The comparison of sequences and determination of percent identity between two sequences is accomplished using a mathematical algorithm. In some embodiments, percent identity between two amino acid sequences is determined using the Needleman and Wunsch, J. Mol. Biol., 48:444-453, 1970) algorithm, which has been incorporated into the GAP program in the GCG software package, using either a Blosum 62 matrix or a PAM250 matrix, and a gap weight of 16 and a length weight of 1. The percent identity between two nucleotide sequences can be determined using the GAP program in the GCG software package using a NWSgapdna CMP matrix, a gap weight of 40, and a length weight of 1.

In general, percent identity between amino acid sequences referred to herein can be determined using the BLAST 2.0 program, which is available to the public on the Internet at ncbi.nhn.nih.gov/BLAST. In some embodiments, sequence comparison is performed using an ungapped alignment and using the default parameters (Blosum 62 matrix, gap existence cost of 11, per residue gap cost of 1, and a lambda ratio of 0.85). In some embodiments, default parameters are used. The mathematical algorithm used in BLAST programs is described in Altschul et al., Nucleic Acids Research 25:3389-3402, 1997. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a target protein is generally replaced with another amino acid residue from the same side chain family. Alternatively, mutations can be introduced randomly along all or part of a target protein coding sequence, such as by saturation mutagenesis, and the resultant mutants can be screened for target protein biological activity to identify mutants that retain activity. The encoded protein can be expressed recombinantly and the activity of the protein can be determined.

Also provided herein are chimeric or fusion proteins, wherein said chimeric or fusion proteins comprise at least a portion of a target protein, antibody to a target protein, or detectable polypeptide.

Nucleic acids that encode a target protein or fragment or variant thereof (e.g., a human target protein) are provided, wherein such nucleic acid(s) are operably linked to a promoter, e.g., in an expression vector. Such nucleic acids are of use, e.g., to express the protein for therapeutic or research, e.g., in order to produce the protein in vivo (e.g., in a human subject for therapeutic purposes or in an animal model) or in vitro (e.g., to produce the protein for therapeutic, research, or other purposes).

### Antibodies

The invention provides antibodies that bind to a target protein identified herein or are otherwise of use in one or more inventive method described herein. “Antibodies” as used herein can be polyclonal, monoclonal, humanized, anti-idiotypic, chimeric or single chain antibodies, and Fab, F(ab′)2 and Fab expression library fragments, scFV molecules, and epitope-binding fragments thereof, in various embodiments of the invention. A target protein, or a fragment thereof, can be used as an immunogen to generate antibodies using standard techniques for polyclonal and monoclonal antibody preparation. The full-length polypeptide or protein can be used or, alternatively, antigenic peptide fragments can be used as immunogens. The antigenic peptide of a protein typically comprises at least 8 (e.g., at least 10, 15, 20, or 30) amino acid residues of the amino acid sequence of a target protein, and encompasses an epitope of a target protein such that an antibody raised against the peptide forms a specific immune complex with the polypeptide.

An immunogen typically is used to prepare antibodies by immunizing a suitable subject (e.g., rabbit, goat, mouse or other mammal). An appropriate immunogenic preparation can contain, for example, a recombinantly expressed or a chemically synthesized polypeptide. The preparation can further include an adjuvant, such as Freund's complete or incomplete adjuvant, or similar immunostimulatory agent. Polyclonal antibodies can be prepared as described above by immunizing a suitable subject with a target protein as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, Nature, 256:495-497, 1975, the human B cell hybridoma technique (Kozbor et al., Immunol. Today, 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known). Hybridoma cells producing a monoclonal antibody can be detected by screening the hybridoma culture supernatants for antibodies that bind the polypeptide of interest, e.g., using a standard ELISA assay. As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide of interest. Kits for generating and screening phage display libraries are commercially available. Additionally, recombinant antibodies, such as chimeric and humanized monoclonal antibodies, including both human and non-human portions, which can be made using standard recombinant DNA techniques, are provided herein. Such chimeric and humanized monoclonal antibodies can be produced by recombinant DNA techniques known in the art. Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a target protein. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgQ IgA, and IgE antibodies. For an overview of this technology for producing human antibodies, see Lonberg and Huszar (Int. Rev. Immunol., 13:65-93, 1995). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., Biotechnology, 12:899-903, 1994).

An antibody directed against a target protein can be used to detect the polypeptide (e.g., in a cellular lysate or cell supernatant) to evaluate its abundance and pattern of expression. The antibodies can also be used diagnostically to monitor protein levels in tissue as part of a clinical testing procedure, e.g., for example, to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Antibodies can also be used to modulate the activity of a target protein, e.g., for research or therapeutic purposes. Antibodies often inhibit their target but, depending on the epitope to which they bind, could activate their target, or antibodies that bind to an endogenous inhibitor of the target can enhance the activity of the target. In some embodiments, a target protein has one or more particular biochemical activities and/or biological functions, and a compound that modulates such function(s) is identified. For example, the target protein may be an enzyme that catalyzes a biochemical reaction or may be a regulator (e.g., an activator or inhibitor) of an enzyme. Enzymes may be classified broadly as oxidoreductases, transferases, hydrolases, lyases, isomerase, or ligases. (See, e.g., Enzyme Nomenclature: Recommendations of the Nomenclature Committee of the International Union of Biochemistry and Molecular Biology on the Nomenclature and Classification of Enzymes by the Reactions they Catalyse, available at http://www.chem.qmul.ac.uk/iubmb/enzyme/). Exemplary enzymes include, e.g., kinases, phosphatases, GTPases. Exemplary enzyme regulators include, e.g., guanine nucleotide exchange factors.

Compounds can be tested for their ability to modulate one or more enzymatic activities or enzyme regulatory activities mediated by a target protein described herein. One of skill in the art will be aware of suitable assays to identify modulators of such enzymes. For example, kits for performing a wide variety of enzyme assays, e.g., assays for kinase, phosphatase, ATPase, GTPase, and ubiquitinating activity, among others, are commercially available. In some embodiments, a suitable assay involves (i) incubating the target protein, a suitable substrate, and a test compound under appropriate conditions; and (ii) detecting production of one or more products of the reaction, wherein an increased amount of reaction product(s) as compared with the amount of reaction product(s) that would be expected in the absence of the test compound indicates that the test compound enhances activity of the enzyme, and a decreased amount of reaction product(s) as compared with the amount of reaction product(s) that would be expected in the absence of the test compound indicates that the test compound inhibits activity of the enzyme. Detecting can be qualitative or quantitative in various embodiments. “Appropriate conditions” refer to conditions under which the target protein would (in the absence of the test compound), effectively catalyze the reaction, so that reaction product(s) would appear in a detectable amount in a reasonable time frame for detection (e.g., seconds, minutes, hours). Appropriate conditions may include, e.g., an appropriate temperature, pH, salt concentration; presence of one or more organic or inorganic co-factors and/or energy source(s), etc. It will be understood that many enzymes can often tolerate a range of different conditions, e.g., a range of values for temperature, pH, salt concentration, etc. In some embodiments, an enzyme is a multisubunit protein, in which case the assay components can include at least the catalytic subunit(s) and, optionally, one or more additional subunits of the enzyme, e.g., those subunit(s) that form a complex with the catalytic subunit in nature. Optionally, at least one of the substrates is labeled (e.g., with a radiolabel, colorimetric, fluorescent, luminescent, or enzymatic label, etc., to facilitate detection of the reaction or reaction product(s) or a colorimetric, fluorescent, luminescent signal is generated upon occurrence of the reaction. In some embodiments, the enzymatic reaction converts a non-fluorescent moiety into a fluorescent moiety, which can then be detected. In some embodiments, a substrate contains a fluorophore and a dark quencher, wherein the dark quencher absorbs energy from the fluorophore and dissipates the energy as heat. Upon cleavage of the substrate, the dark quencher becomes separated from the fluorophore, so that the energy from the fluorophore is no longer absorbed and can be detected, thereby providing a means to detect the reaction.

Functional assays can also be used to identify modulators of expression or activity of a gene or protein identified herein or that modulate a biological process in which a gene or protein identified herein is involved. For example, high content screens can involve use of automated imaging, cell sorting, etc., to analyze localization of a protein or alterations in a process in which a protein identified herein is involved. For example, PICALM is involved in clathrin-mediated endocytosis and intracellular trafficking of various proteins, including the synaptic vesicle proteinVAMP2 that functions in neurotransmitter release at the presynaptic membrane.

In some embodiments, computationally based drug design and/or virtual screening is used to identify a modulator, based on 2- or 3-dimensional structure(s) of a protein identified herein.

In some embodiments, a screen is performed to identify compounds that affect endocytosis (e.g., clathrin-mediated endocytosis), e.g., that increase flux through the endocytic pathway. A variety of assays for endocytosis can be used. See, e.g., Osborne, A, et al., (2005) Current Protocols in Cell Biology Unit Number: Unit 11.18; Knisely, J et al. (2008) Methods in Molecular Biology, 457: 1-14. In some embodiments, a labeled derivative of a protein is used, e.g., to facilitate imaging. For example, the protein can be fluorescently labeled (e.g., as a fusion protein comprising a fluorescent polypeptide such as GFP). In some embodiments, a compound that selectively accumulates in endosomes or a protein that localizes to endosomes is used.

In some embodiments of the invention, a target gene encodes a phosphatase, e.g., a protein phosphatase (e.g., PP2A) or a phosphoinositide phosphatase (e.g., SYNJ1). Assays for measuring phosphatase activity are known in the art. For example, such assays can comprise incubating a substrate comprising a phosphate group together with a phosphatase (e.g., PP2A or SYNJ1) under conditions suitable for the phosphatase to dephosphorylate the substrate. After a suitable time period, the amount of dephosphorylation can be assessed, e.g., by measuring free phosphate (e.g., using a malachite green assay) and/or by measuring the amount of dephosphorylated substrate. The dephosphorylated substrate can be detected, e.g., using antibodies that specifically recognize either the phosphorylated or dephosphorylated form of the substrate or based on the difference in size caused by dephosphorlyation (which may be evident as altered migration on a gel), or using mass spectrometry. See, e.g., Keen et al. (2005) J. Biol. Chem. 280(33):29519; Mitsuhashi et al. (2005) Mol. Cell. Biochem. 269(1-2): 183; Mani et al. (2007) Neuron. 56(6): 1004-1018. Such phosphatase activity assays or variants thereof can be used to identify compounds that inhibit or enhance the activity of a phosphatase (e.g., PP2A or SYNJ1) described herein.

In some embodiments, a target gene encodes a protein kinase, e.g., MAP2K4. MAP2K4 (also known as MKK4) is a component of stress activated MAP kinase signaling modules and directly phosphorylates and activates the c-Jun N-terminal kinase (JNK) and p38 families of MAP kinases. MAP2K4 is itself activated by various Map kinase kinase kinases (MKKKs), which phosphorylate the Ser/Thr residues of MAP2K4 in the Ser-11e-Ala-Lys-Thr motif located in the T-loop of the kinase domain. For example, MEKK1 and MEKK4 activate MKK4. Assays for measuring kinase activity are well known in the art. See, e.g., Lawler, S. et al. (1998) Curr. Biol. 8, 1387-1390; Fleming, Y. et al. (2000) Biochem. J. 352:145-154. Such assays or variants thereof can be used to, e.g., identify compounds that inhibit or enhance the activity of a protein kinase such as any of those described herein. It will be appreciated that a variety of high throughput kinase assays are known in the art and could be used in the practice of the invention. See, e.g., von Ahsen O, et al. (2005) Chembiochem. 6(3):481-90. In some embodiments, a kinase inhibitor targets the ATP binding site of the kinase. In some embodiments, an inhibitor of MAP2K4 comprises a compound that inhibits an interaction between MAP2K4 and a MKKK. For example, it has been reported that mammalian MAPKKs, including MKK4, dock with their activating kinase(s) through a domain termed the domain for versatile docking (DVD), which corresponds to amino acids 364-387 in MKK4 (Takekawa et al., (2005) Molecular Cell 18(3): 295-306). In some embodiments, an inhibitor of MAP2K4 blocks docking of an activating kinase to the MAP2K4 DVD, thereby inhibiting activation of MAP2K4.

RABGEF1 serves as a guanine nucleotide exchange factor (GEF) for Rab5 proteins (Carney D S, et al. (2006) Trends Cell Biol. 16(1):27-35). As known in the art, Rab proteins (including Rab5) comprise a family of small GTPases whose localization and cycling between active (GTP bound) and inactive (GDP bound) conformations depends on regulatory factors that modulate membrane association, nucleotide binding, and GTP hydrolysis. Rab GTPases localized to membranes are activated by guanine nucleotide exchange factors (GEFs) and subsequently interact with effectors to facilitate processes such as vesicle budding, cargo sorting, and transport as well as the tethering, docking, and fusion of vesicles with acceptor membranes. For example, RABGEF1 (GEF) activates Rab5, converting Rab5-GDP to Rab5-GTP. GTP hydrolysis is accelerated by GTPase-activating proteins (GAPs) to complete the cycle. Rab proteins possess a third class of regulatory proteins, termed guanine nucleotide dissociation inhibitors (GDIs) that prevent GDP-GTP exchange (maintaining the small GTPase in an off-state) and thus counteract the effect of GEFs. In some embodiments, screens are conducted to identify compounds that modulate, e.g., inhibit, the physical interaction between RABGEF1 and a GTPase target of RABGEF1, e.g., Rab5 protein, or that modulate the activity of the GTPase itself or its localization. For example, compounds that inhibit or enhance membrane localization of Rab5 can be identified.

In some embodiments, a human protein identified herein as a homolog of a yeast suppressor or enhancer is introduced into non-human cells, e.g., yeast cells, and, optionally, such cells are used for performing a screen to identify compounds that modulate activity of the human protein. Optionally, the gene encoding the counterpart homologous protein in said cells may be disabled or deleted. For example, in one embodiment, a yeast gene identified herein is deleted, and the counterpart human gene is expressed in the cell. For example, the counterpart human gene can be integrated into the yeast genome, optionally under control of an inducible promoter. In some embodiments, the cells express a toxicity-inducing amount or form of amyloid beta. In some embodiments, the cells are from a transgenic non-human animal, wherein the transgenic non-human animal comprises (i) a transgene that encodes a toxicity-inducing form of amyloid beta and/or, upon expression, results in production of a toxicity-inducing amount or form of amyloid beta; and/or (ii) a transgene that encodes a human protein identified herein as a homolog of a yeast suppressor or enhancer of amyloid beta mediated toxicity.

In some embodiments, screens and/or further characterization of compounds is/are carried out using vertebrate cells or tissues, e.g., mammalian (e.g., mouse, rat, or human) cells or tissues. In some embodiments, the vertebrate cells are neurons, e.g., neurons from a region of the brain such as the hippocampus. In some embodiments, the neurons are pyramidal neurons. In some embodiments the neurons are cholinergic neurons. In some embodiments the neurons are glutamatergic neurons. In some embodiments, the tissues comprise brain tissue, e.g., brain slices, e.g., hippocampal brain slices.

### Compounds

Compounds to be screened or identified using any of the methods described herein can include various chemical classes, though typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

In alternative embodiments, compounds can also include biomolecules including, but not limited to, peptides, polypeptides, peptidomimetics (e.g., peptoids), amino acids, amino acid analogs, saccharides, fatty acids, steroids, purines, pyrimidines, derivatives or structural analogues thereof, polynucleotides, nucleic acid aptamers, and polynucleotide analogs.

Compounds can be identified from a number of potential sources, including: chemical libraries, natural product libraries, and combinatorial libraries comprised of random peptides, oligonucleotides, or organic molecules. Chemical libraries consist of diverse chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997).

Identification of test compounds through the use of the various libraries herein permits subsequent modification of the test compound “hit” or “lead” to optimize the capacity of the “hit” or “lead” to prevent or suppress amyloid beta-induced toxicity and/or amyloid beta-induced aggregation.

The compounds identified above can be synthesized by any chemical or biological method. The compounds identified above can also be pure, or may be in a composition (e.g., a pharmaceutical composition) that contains one or more additional component(s), and can be prepared in an assay-, physiologic-, or pharmaceutically-acceptable diluent or carrier (see below).

### Biomarkers and Predictive Methods; Kits

The invention further provides methods of identifying biomarkers for AD. In some embodiments, a method comprises identifying a polymorphism or mutation in the sequence of a gene identified herein or an alteration in the expression of a gene identified herein or the activity of its encoded protein; and (b) determining whether the polymorphism, mutation, or alteration is correlated with development of AD. Such methods may, for example, utilize available genotyping, clinical and pathological data from studies, e.g., epidemiological studies, of aging, cognition, and AD. Collections of data and samples and methods (e.g., statistical methods) known in the art can be used to identify and/or confirm correlations. Once a correlation is confirmed, it may be used, e.g., in the identification of subjects who may benefit from therapy, e.g., therapy using a compound identified as described herein or a therapy known in the art to be of use for treating AD. In some embodiments, a genome-wide association study is performed. In some embodiments, a case-control study is performed. In some embodiments, one or more SNPs available in the dbSNP database (http://www.ncbi.nlm.nih.gov/projects/SNP) in a gene identified herein are evaluated.

The invention further provides methods for identifying subjects who are at increased risk of developing an amyloid beta mediated disease, e.g., AD. Certain of the methods comprise determining whether a subject exhibits altered sequence, expression, or activity of a gene identified herein and/or of a protein encoded by a gene identified herein, as compared with a reference, wherein altered sequence, expression, or activity of the gene or of a protein encoded by the gene indicates that the subject is at increased risk of developing or having AD.

In some embodiments, the gene is selected from human genes encoding XRN1, NET1, POMT2, PASK, SEL1L2, SNX8, MAP2K4, PKN2, LARP1, PICALM, PPP2R5C, MARK4, TEAD2, ADSSL1, XPO1, SH3KBP1, MITF, LARP1B, DAPK1, SYNJ1, FBXL2, and RABGEF1, e.g., the gene is selected from human genes encoding PICALM, PPP2R5C, ADSSL1, XPO1, SH3KBP1, SYNJ1, FBXL2, RABGEF1, XRN1, POMT2, SNX8, and MAP2K4. In some embodiments, at least two of the foregoing genes (or their encoded proteins) are assessed. In some embodiments, increased expression of a gene identified as an enhancer of amyloid beta mediated toxicity and/or increased activity of a protein encoded by an enhancer indicates that a subject is at increased likelihood of developing or having AD. In some embodiments, decreased expression of a gene identified as a suppressor of amyloid beta mediated toxicity and/or decreased activity of a protein encoded by an enhancer indicates that a subject is at increased likelihood of developing or having AD. A reference sequence or reference level may, e.g., reflect the sequence or level of expression or activity typically found in control subjects. For example, a reference level may be an average level among control subjects or a range around that level (e.g., a range within which at least 80%, 90%, or 95% of control subjects fall. In some embodiments, control subjects are individuals who are not suffering from AD and do not have signs or symptoms associated with cognitive impairment or other characteristics that might indicate a significant likelihood of developing AD. Control subjects may be matched for a variety of characteristics such as age, sex, general health status (e.g., absence of cardiovascular disease), etc. Historical controls can be used. In some embodiments, the methods can be used to monitor progression of the disease and/or response to therapy. In some embodiments, a result, e.g., a difference in level of expression or activity as compared with a reference level, is statistically significant. For example, a result can have a P value of less than 0.05 or less than 0.01 in various embodiments.

An alteration in gene expression can be caused, e.g., by a mutation or polymorphism in a coding region or non-coding region (e.g., a regulatory region) of a gene or an alteration (increase or decrease) in copy number or an epigenetic change (e.g., altered DNA methylation or altered histone modification (e.g., altered acetylation or methylation). A mutation can be, e.g., an insertion, deletion, or substitution. A polymorphism may be a single nucleotide polymorphism. An alteration in activity of a protein may be caused by a mutation in a coding region of the gene, wherein the mutation alters the sequence of the encoded protein. The invention encompasses analyzing a sample comprising DNA, mRNA, or protein obtained from a subject (e.g., from lymphocytes), to assess the sequence (e.g., to determine the identity of one or more nucleotides or amino acids), expression level, and/or activity of a gene identified herein or its encoded protein and, optionally, comparing said sequence, expression, and/or activity level with a reference sequence or reference level of expression or activity. Methods known in the art including, but not limited to, methods mentioned herein, can be used to assess gene sequence, expression level, and/or activity of a protein. For example, genotyping can be performed using DNA microarrays (e.g., available from Affymetrix, Illumina, Agilent), real-time PCR, and/or sequencing (e.g., high throughput sequencing, such as Illumina/Solexa technology or the SOLiD™ system). Epigenetic changes can be detected, e.g., using bisulfite sequencing, ChIP-on-chip and/or ChIP-seq. Alterations in protein level and/or modification can be detected, e.g., using immunoassays or mass spectrometry. In some embodiments, an alteration in activity of a protein may be caused by an alteration in post-translational modification of the protein, e.g., an alteration in phosphorylation, glycosylation, lipidation, alkylation, ubiquitination, sumoylation, etc. Thus in some embodiments the invention encompasses detecting such modifications, or alterations therein. In some embodiments of the invention, a subject who exhibits decreased expression of a gene and/or decreased expression of a protein identified herein (e.g., a suppressor of amyloid beta toxicity) is treated or selected as a candidate for treatment with a compound that increases expression or activity of the protein. In some embodiments of the invention, a subject who exhibits increased expression of a gene and/or increased expression of a protein identified herein (e.g., an enhancer or of amyloid beta toxicity) is treated or selected as a candidate for treatment with a compound that decreases expression or activity of the protein.

Further provided are kits containing reagent(s) useful for detecting sequence, expression, or activity of a gene identified herein and/or of a protein encoded by a gene identified herein and/or for detecting alteration in any of the foregoing. Such reagents can comprise, e.g., nucleic acid probes or primers for detecting gene sequence or expression or alteration therein (optionally attached to a support such as a chip or beads), antibodies for detecting protein level, size, modification, or alteration therein. The kits can comprise control or reference samples, instructions for performing the assay and/or for interpreting results, etc. In some embodiments, a kit is adapted specifically for identifying subjects at risk of or suffering from an amyloid beta mediated disease and/or for assessing a selected set of genes and/or proteins identified as potentially harboring mutation(s), polymorphism(s), or epigenetic changes that are risk factors for an amyloid beta mediated disease. For example, if the kit provides for assessment of multiple genes or proteins, the proportion of genes or proteins implicated or confirmed as risk factors for an amyloid beta mediated disease is greater than would be expected by chance. In some embodiments, at least 10%, 20%, 30%, 40%, 50%, 60%, 70%, 80%, 90% or 100% of the genes or proteins to be assessed are implicated or confirmed as risk factors for an amyloid beta mediated disease. In some embodiments, the kit components are enclosed in one or more containers that are labeled or packaged with information or instructions that indicate, e.g., that the kit is useful for identifying or assessing subjects at risk of or suffering from an amyloid beta mediated disease.

Detection of an alteration in sequence, expression, or activity of a gene or protein identified herein may be used together with additional information to assess the likelihood that a subject will develop or has AD. Such information may be genetic information (e.g., presence of particular risk allele(s)), imaging information, biochemical information (e.g., measurement of amyloid beta or tau levels in CSF), neuropsychological information, and/or clinical information.

### Pharmaceutical Compositions and Methods of Treatment

A compound that is found to prevent or suppress amyloid beta-induced toxicity or the formation of amyloid beta aggregates in a cell can be formulated as a pharmaceutical composition, e.g., for administration to a subject to treat a neurodegenerative disease such as Alzheimer's disease.

A pharmaceutical composition typically includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge et al., J. Pharm. Sci. 66:1-19, 1977).

The compound can be formulated according to standard methods. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

In one embodiment, a compound that prevents or suppresses amyloid beta-induced toxicity and/or amyloid beta aggregate formation in a cell can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

The pharmaceutical compositions may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The preferred form can depend on the intended mode of administration and therapeutic application. In some embodiments, compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating a compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of a compound plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the compound can be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

A compound identified as one that prevents or suppresses amyloid beta-induced toxicity and/or amyloid beta aggregate formation in a cell can be modified, e.g., with a moiety that improves its stabilization and/or retention in circulation, e.g., in blood, serum, or other tissues, e.g., by at least 1.5, 2, 5, 10, or 50 fold. The modified compound can be evaluated to assess whether it can reach treatment sites of interest (e.g., locations of aggregate amyloid beta) such as can occur in a cell in a subject with a neurodegenerative disease such as Alzheimer's disease (e.g., by using a labeled form of the compound).

For example, the compound can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, a compound can be conjugated to a water soluble polymer, e.g., a hydrophilic polyvinyl polymer, e.g., polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides.

When the compound is used in combination with a second agent (e.g., any additional therapies for Alzheimer's disease or other disorders associated with amyloid beta mediated toxicity and/or the formation, deposition, accumulation, or persistence of amyloid beta aggregates), the two agents can be formulated separately or together. For example, the respective pharmaceutical compositions can be mixed, e.g., just prior to administration, and administered together or can be administered separately, e.g., at the same or different times.

Compounds described herein and those identified as described herein can be used to treat a subject (e.g., a human subject) that is at risk for or has a disorder associated with amyloid beta mediated toxicity and/or the formation, deposition, accumulation, or persistence of amyloid beta aggregates, e.g., amyloid beta oligomers and/or dimers. In certain embodiments, the disorder is Alzheimer's disease, Down's Syndrome, Fragile X syndrome, systemic amyloidosis, or sporadic inclusion body myositis. Methods of identifying an individual at risk for or having such disorders are known in the art. For example, AD can be diagnosed based on, e.g., patient history (e.g., memory loss) clinical observations, the presence of characteristic neurological and neuropsychological features, and the absence of other conditions that might be responsible for the foregoing. Imaging techniques such as computed tomography (CT), magnetic resonance imaging (MRI), single photon emission computed tomography (SPECT), or positron emission tomography (PET) can be of use. Diagnosis can be confirmed by post-mortem examination of brain material. Exemplary criteria for diagnosis of AD are found in the Diagnostic and Statistical Manual of Mental Disorders (DSM)-IV (text revision, 2000) or DSM-V and the National Institute of Neurological and Communicative Disorders and Stroke (NINCDS)-Alzheimer's Disease and Related Disorders Association (ADRDA) criteria (McKhann G, et al. (1984) Neurology 34 (7): 939-44), e.g., as updated (Dubois B, et al. (2007) Lancet Neurol 6 (8): 734-46). Analysis of cerebrospinal fluid (CSF) for various biomarkers, e.g., amyloid beta or tau proteins (e.g., total tau protein and phosphorylated tau) and/or imaging (e.g., PET imaging) with labeled compounds that bind to amyloid beta deposits (e.g. 11C-labeled Pittsburgh Compound-B (11C-PIB) or 18F-AV-45 (flobetapir F18)) can be used to predict the onset of AD, e.g., to identify individuals who have a significant likelihood of progressing to AD in the future (e.g., within the next two years). Such imaging methods may also be of use in the instant invention to assess the in vivo effect of compounds identified herein and/or identified using an inventive screening assay. In some embodiments, a subject has a mutation in a gene encoding amyloid precursor protein (APP), presenilin 1, or presenelin 2. In some embodiments, the mutation increases the production of Aβ42 or alters the ratio of Aβ42 to Aβ40. In some embodiments the subject has at least one copy of the ε4 allele of the apolipoprotein E (APOE) gene.

Downs' Syndrome can be diagnosed based on presence of trisomy 21.

Fragile X Syndrome is caused by expansion of a trinucleotide gene sequence (CGG) on the X chromosome that results in a failure to express the protein coded by the FMR1 gene, which encodes FMRP. It may be suspected based on the presence of characteristic signs and symptoms, with diagnostic confirmation from genetic testing.

Sporadic inclusion-body myositis (sIBM) is the most common acquired muscle disease in individuals over the age of 50 (although it may also occur in younger individuals). Clinically, sIBM frequently initially causes a selective pattern of muscle weakness involving the forearm flexor and quadriceps femoris muscles, eventually evolving into a syndrome of diffuse, progressive, asymmetric, proximal, and distal weakness and muscle atrophy. The clinical diagnosis of sIBM is aided by electromyography (EMG) and can be confirmed by muscle biopsy, which is considered the “gold standard” for diagnosis (Dalakas, M. (2006) Nature Clinical Practice Neurology, 2: 437-447).

Pathologically, sIBM is characterized by intramuscular inflammatory and degenerative features. The inflammatory component typically includes endomysial infiltrates composed predominantly of CD8+ T cells. Expression of MHC class I antigens in non-necrotic muscle fibers can typically be detected. Characteristic findings in myofibers affected by sIBM are vacuolization, abnormal mitochondria, abnormal myonuclei, and Congo red-positive amyloid deposits (Dalakas, M. supra). Deposits of Aβ and various other proteins are typically evident (Askanas V, et al. (2009) Brain Pathol. 19(3):493-506). Aβ accumulation may occur as a result of increased synthesis and/or abnormal processing of APP in sIBM muscle. Thus, methods and compositions for treating a subject at risk of (or susceptible to) an amyloid beta mediated disease are described herein. For example, an individual who is at risk of developing AD and/or has signs suggesting that he or she will develop AD can be treated with the compounds and methods described herein.

As used herein, the term “treatment” is defined as the application or administration of a therapeutic compound to a patient, or application or administration of a therapeutic compound to a subject (e.g., a human subject, who may be referred to as a “patient”) who has a disease (or other medically recognized disorder or syndrome), a symptom of disease or a predisposition toward a disease (e.g., one or more risk factors associated with the disease), with the purpose to cure, heal, alleviate, relieve, alter, remedy, ameliorate, improve, or affect (in a manner beneficial to the subject) the disease, the symptoms of disease or the predisposition toward disease. In some embodiments, treatment is prophylactic, i.e., it is administered to a subject who has not developed the disease (and who may or may not have a predisposition to develop the disease) with an intent to delay, prevent, or reduce the likelihood that the subject will develop the disease or reduce the severity should the subject develop the disease. Compounds may also or alternately be administered to a subject for purposes of testing, research, or diagnosis and/or may be contacted with an isolated tissue, cells, or cell line from a patient, e.g., for purposes of testing, research, diagnosis, or with an intent to subsequently administer the isolated tissue, cells, or cell line to the subject for treatment.

The invention provides methods for preventing in a subject (e.g., a human), an amyloid beta mediated disease by administering to the subject a target protein or a compound that modulates target protein expression or at least one target protein activity. Subjects at risk for a disease that is caused or contributed to by aberrant or unwanted target protein expression or activity can be identified by, for example, any or a combination of diagnostic or prognostic assays as described herein. Administration of a prophylactic compound can occur prior to the manifestation of symptoms characteristic of the disease, such that the disease or disorder is prevented or, alternatively, delayed in its progression. Methods known in the art can be used to determine the efficacy of the treatment. The appropriate compound used for treating the subject can be determined based on screening assays described herein. Some cases of AD (or other amyloid beta mediated diseases) may be caused, at least in part, by an abnormal level of a target gene product, or by the presence of a target protein exhibiting abnormal activity. In some aspects of the invention, altering the level and/or activity of such gene product(s) will bring about the amelioration of disorder symptoms. For example, an abnormally high level of a target gene product or an abnormally high level of activity of a target protein can be reduced and/or an abnormally low level of a target gene product or an abnormally low level of activity of a target protein can be increased. Accordingly, the modulation in the level and/or activity of such gene products will bring about the amelioration of disorder symptoms. Thus in some embodiments, treatment of an amyloid beta mediated disorder can be accomplished by techniques that inhibit the expression or activity of selected target gene products (e.g., enhancers of amyloid beta toxicity). In some embodiments, treatment of an amyloid beta mediated disorder can be accomplished by techniques that enhance the expression or activity of selected target gene products (e.g., suppressors of amyloid beta toxicity). For example, compounds, e.g., an agent identified using one or more of the assays described above, that exhibits negative modulatory activity (e.g., that inhibits gene expression or protein activity), can be used as described herein to prevent and/or ameliorate symptoms of an amyloid beta mediated disorder. Such molecules can include, but are not limited to, peptides, small organic or inorganic molecules, antibodies, siRNA, miRNA, antisense, aptamer, ribozyme, or triple helix molecules. In some embodiments, the compound enhances one or more target protein activities. Examples of such compounds can include, but are not limited to, active target protein (or biologically active fragment or variant thereof), nucleic acid molecules encoding the target protein or encoding a biologically active fragment or variant thereof, antagomirs that inhibit miRNA that would otherwise inhibit expression of the gene, peptides, small organic or inorganic molecules, and antibodies.

Compounds that modulate (inhibit or enhance) target gene expression, synthesis and/or activity can be administered to a patient at therapeutically effective doses to prevent, treat, or ameliorate an amyloid beta mediated disorder. A therapeutically effective dose can be an amount of the compound sufficient to result in amelioration of a one or more symptom(s) or sign(s) of the disorder and/or reduction in rate at which one or more symptom(s) or sign(s) of the disorder increases in severity (e.g., as compared with the rate that would be expected in the absence of therapy). Criteria for assessing efficacy in AD are known in the art. Toxicity and therapeutic efficacy of such compounds can be determined by standard pharmacological procedures. In some embodiments, one or more endpoints or sets of endpoints described in Vellas, B., et al., Lancet Neurology (2008) 7(5): 436-450 may be used. In some embodiments, one or more endpoints or sets of endpoints described in Aisen, P S, et al., Neurology (2010) Dec. 23 epub ahead of print are used for evaluating efficacy in early (predementia) AD. It will be understood that an effective dose is typically administered multiple times, e.g., once or more times daily, weekly, monthly, etc., depending on various factors such as, e.g., its half-life. In exemplary embodiments, a therapeutically effective amount ranges from about 0.001 to 100 mg/kg body weight, e.g., about 0.01 to 25 mg/kg body weight, about 0.1 to 20 mg/kg body weight, about 1 to 10 mg/kg body weight. One in the art will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

In some aspects, methods of the invention include treating an individual afflicted with a disease or disorder characterized by aberrant or unwanted expression or activity of a target protein or nucleic acid molecule. In one embodiment, the methods involve administering a compound (e.g., a compound identified by a screening assay described herein), or combination of compounds that modulate (e.g., up regulates or down regulates) target protein expression or activity. Stimulation of target protein activity may be desirable in situations in which target protein is abnormally downregulated and/or in which increased target protein activity is likely to have a beneficial effect. Inhibition of target protein activity may be desirable in situations in which target protein is abnormally upregulated and/or in which decreased target protein activity is likely to have a beneficial effect. In certain embodiments, one or more compounds (e.g., compounds that modulate expression or activity of different genes or proteins) can be administered, together (simultaneously) or at different times (sequentially).

The following are examples of the practice of the invention. They are not to be construed as limiting the scope of the invention in any way.

### EXAMPLES

### Example 1

### Yeast Model of Amyloid Beta Toxicity

Several yeast strains were generated that enable inducible expression of the human amyloid beta 1-42 peptide. The expression construct used in these studies encodes a fusion polypeptide containing the yeast Kar2p signal sequence at the amino terminus and the human amyloid beta 1-42 peptide at the carboxy terminus. A signal sequence was included in the fusion polypeptide to cause the transport of the human amyloid beta 1-42 peptide to the endoplasmic reticulum within the cell. The polypeptide was expressed in yeast under the control of a galactose-inducible promoter.

The amino acid sequence of the fusion polypeptide encoded by the expression construct is: MFFNRLSAGKLLVPLSVVLYALFVVILPLQNSFHSSNVLVRGDAEFRH DSGYEVREVHHQKLVFFAEDVGSNKGAIIGLMVGGVVIA (SEQ ID NO:1). The yeast Kar2p signal sequence corresponds to amino acids 1-42 of SEQ ID NO:1 and the human amyloid beta 1-42 peptide corresponds to amino acids 43-84 of SEQ ID NO:1.

The nucleotide sequence used in the expression construct is:

(SEQ ID NO: 2)
ACAAGTTTGTACAAAAAAGCAGGCTTCACAAAATGTTTTTCAACAGACTA
AGCGCTGGCAAGCTGCTGGTACCACTCTCCGTGGTCCTGTACGCCCTTTT
CGTGGTAATATTACCTTTACAGAATTCTTTCCACTCCTCCAATGTTTTAG
TTAGAGGTGATGCTGAATTTAGACATGATTCTGGTTATGAAGTTCATCAT
CAAAAATTGGTTTTTTTTGCTGAAGATGTTGGTTCTAATAAAGGTGCTAT
TATTGGTTTGATGGTTGGTGGTGTTGTCATTGCTTAAACCCAGCTTTCTT
GTACAAAGTGGT.

The region of SEQ ID NO:2 encoding the fusion polypeptide of SEQ ID NO:1 corresponds to nucleotides 33 to 284 of SEQ ID NO:1.

Western blot analysis demonstrated that the fusion polypeptide was processed when expressed in yeast cells to yield the human amyloid beta 1-42 peptide. The amount of the A amyloid beta 1-42 peptide detected within a cell correlated with the number of copies of the expression construct that were introduced into the cell.

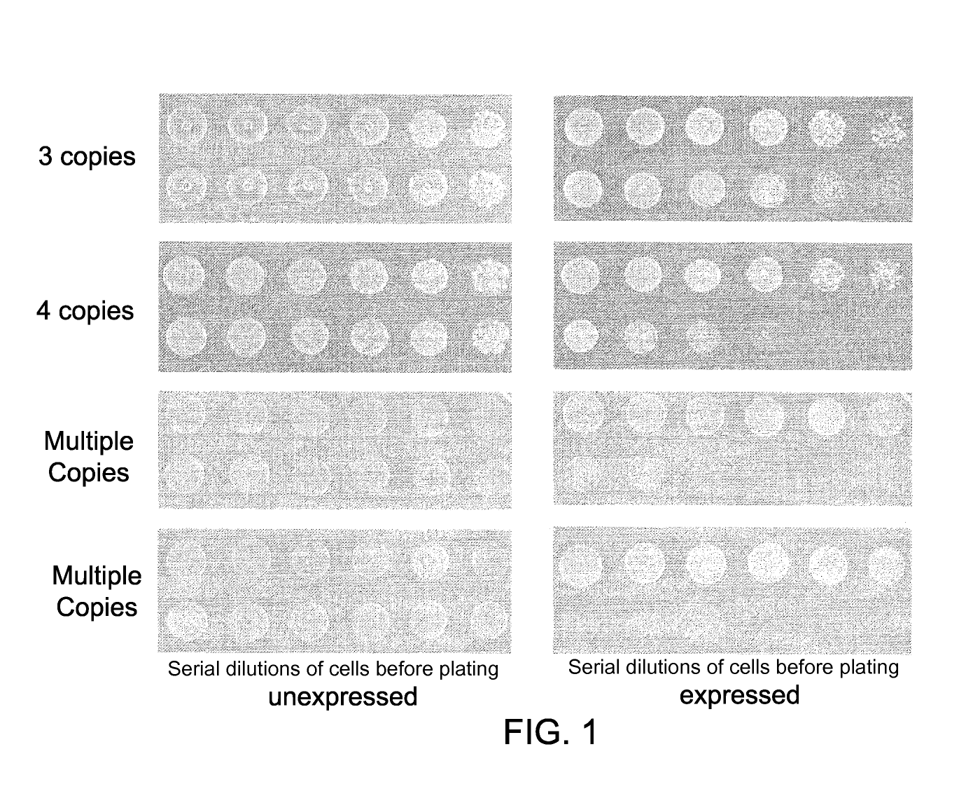

The effect of expression of the fusion polypeptide on yeast cell viability was assessed. Yeast cells were transformed with either an empty vector or a galactose-inducible expression plasmid encoding the fusion polypeptide of SEQ ID NO:1. Serial dilutions of transformants were spotted on glucose or galactose and growth was assessed. Expression of amyloid beta 1-42 peptide (i.e., in transformants grown on galactose plates) was found to be highly toxic to yeast cells (FIG. 1; within each photograph, the empty vector is on the top and the experimental expression vector is on the bottom). The degree of toxicity increased with the number of copies of the expression construct that was introduced into a cell. The yeast cells depicted in the bottom two rows of FIG. 1 (“multiple copies”) contain multiple integrated copies of the expression construct. Although the precise number of copies present in these cells is not known, the enhanced toxicity detected is presumed to be the result of a high copy number of the expression plasmid.

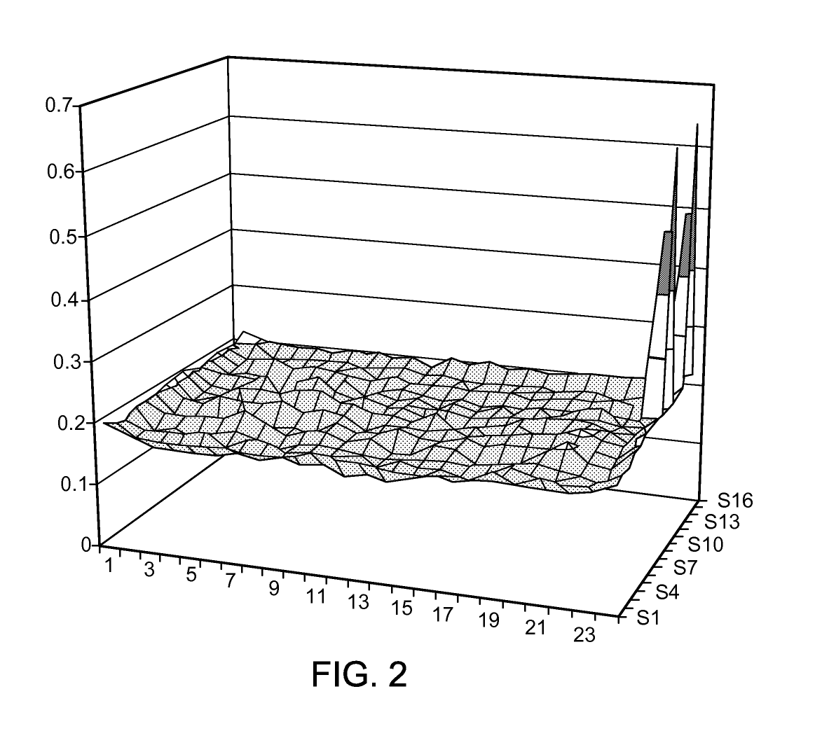

The amyloid beta-expressing yeast cells were used in a screening assay to assess the utility of the recombinant cells in screening for candidate therapeutic compounds. The screen included the following steps: cells were grown in raffinose; cells were diluted to 0.050 in galactose medium (to initiate expression of the expression construct); cells were distributed in a 384 well format; test compounds were added to the wells at a 10 uM final concentration; cell were grown for 48 hours; and the plates were read at A600. The screen identified two wells on the plate as exhibiting growth rescue in the presence of a test compound (FIG. 2). Each of these two wells contained the identical combination of clioquinol and iron (at concentrations of 5 uM each). Clioquinol is a known candidate compound for the treatment of Alzheimer's disease. The results of this screening assay demonstrate that yeast can be used as a model system for amyloid beta-induced cellular toxicity and that candidate therapeutic compounds can be identified that alleviate this toxicity.

### Example 2

### Detection of Aβ 1-42 in the Yeast Secretory System

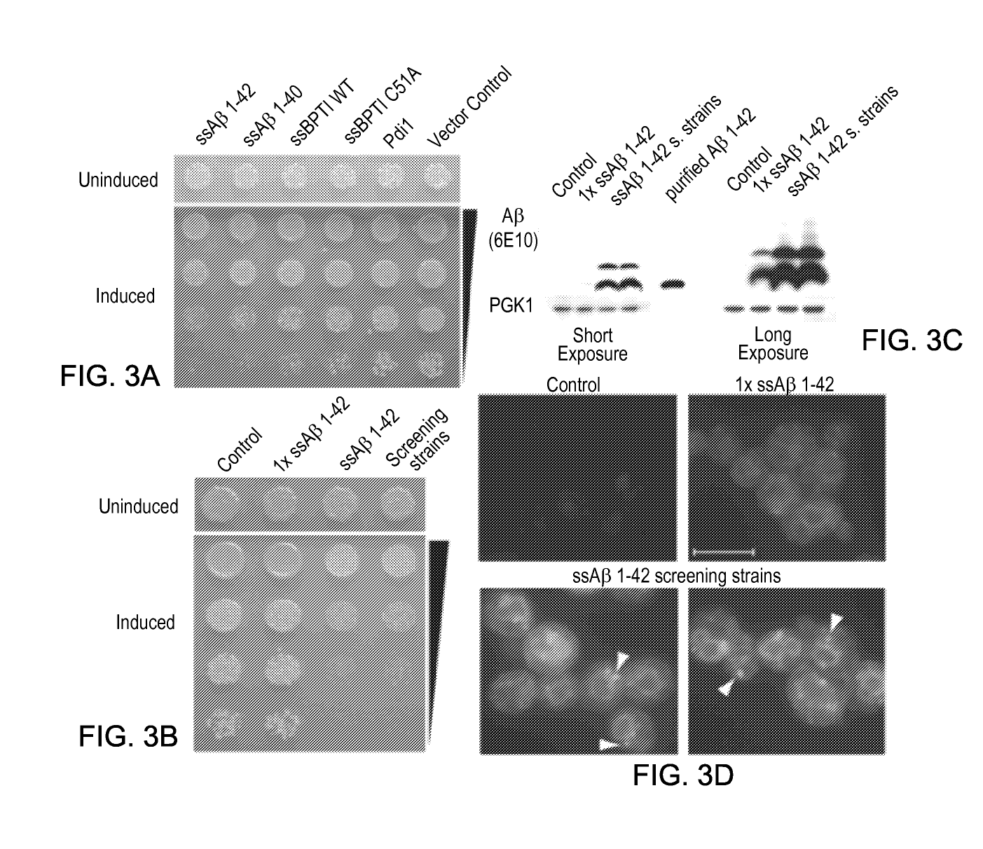

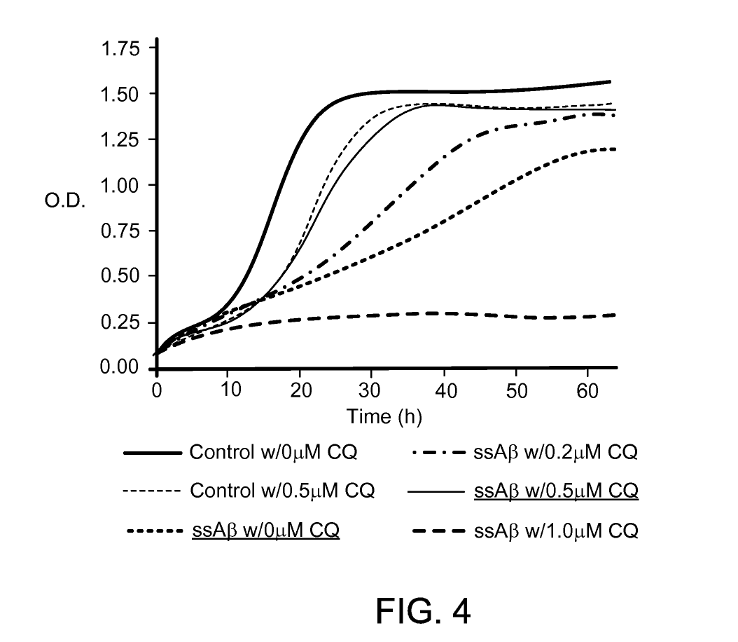

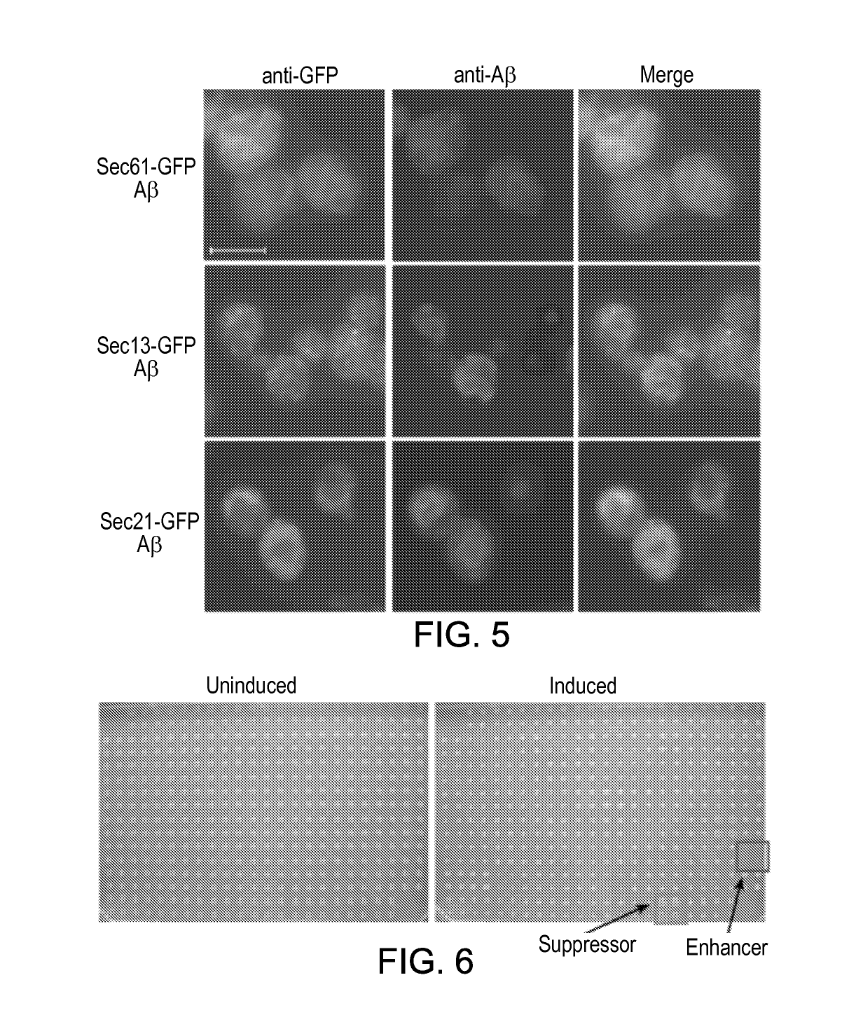

As mentioned above, the integrated constructs resulted in generation of the correct peptide and the formation of higher molecular weight species likely representing Aβ oligomers and dimers. Immunostaining confirmed the localization of Aβ in the secretory system (FIG. 3D and FIG. 5).

### Example 3

### Further Analysis of Clioquinol Rescue

To further test the effect of clioquinol, a control strain and a strain expressing toxic levels of Aβ were grown in liquid culture with various concentrations of the compound. Clioquinol improved the growth of the Aβ expressing strain in a dose dependent manner. At the optimal concentration growth was nearly as robust as that of the control strain (FIG. 3). Importantly, clioquinol did not diminish GAL1-induced expression, as measured by flow cytometry of GAL controlled YFP (data not shown). The most effective concentration of clioquinol varied from 0.5 to 0.8 uM.

### Example 4

### Screen for Genetic Modifiers of Aβ Toxicity

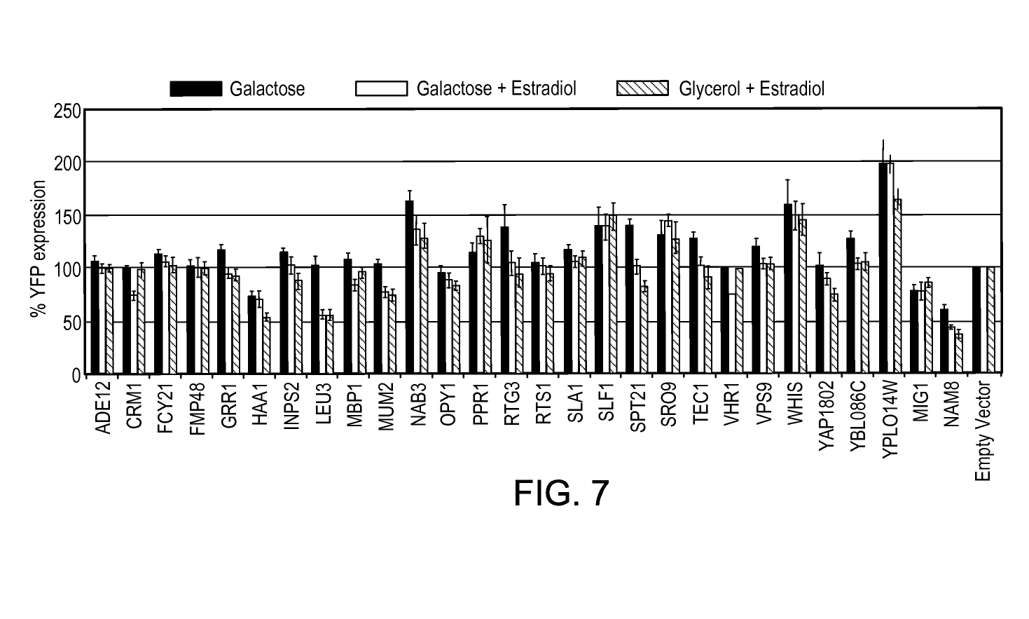

A screen was performed to identify genetic modifiers of Aβ toxicity. To do so, a strain expressing toxic levels of ssAβ 1-42 was transformed with an overexpression library consisting of 5532 plasmids carrying individual ORF clones (out of the 6080 ORFs in yeast) under the control of the GAL1 promoter. The transformed strains were spotted and grown on media that result in different levels of mitochondrial respiration or a non-inducing control media. Putative enhancers and suppressors were identified based on their ability to decrease or increase colony growth (FIG. 6). Hits were retested in independent screening strains and filtered to remove genes that affect GAL-mediated expression (FIG. 7) or affected growth in the absence of Aβ expression. To do so, putative hits were selected and transformed into a strain expressing YFP under control of the GAL promoter. YFP levels were measured using flow cytometry, while yeast growth was examined by spotting a dilution series onto plates with the 4 screening conditions. Ultimately, we identified 23 suppressors and 17 enhancers of Aβ toxicity (Table 1).

The screen hits comprised a wide range of cellular functions. Only a few modifiers were affected by the state of respiration. Twelve had clear human homologs (homology determined using HomoloGene or based on analogous functionality (SLA1-SH3KBP1) (Tables 1, 2, and 3). Strikingly, three of these twelve have functions related to clathrin-mediated endocytosis (YAP1802, SLA1 and INP52; P=3.89e-4) and seven with diverse roles are functionally associated with the cytoskeleton (YAP1802, SLA1, INP52, CRM1, GRR1, KEM1 and RTS1; P=6.06e-8).

Several of the human homologs of these yeast hits have strong connections to AD or Aβ 1-42 toxicity. PICALM, the human homolog of YAP1802, is a well-confirmed risk factor for sporadic AD (Harold et al., Nat Genet (2009) 41:1088; Lambert et al., Nat Genet (2009) 41:1094; Bertram, et al. (2009) Nat Genet. 39:17). PICALM facilitates clathrin assembly and endocytosis (Tebar et al. Mol. Biol. Cell (1999) 10: 2687-2702). Our cells express Aβ 1-42 directly. Thus, our findings not only establish a link between PICALM and Aβ itself, but also implicate normal PICALM function in protection against Aβ 1-42 toxicity.

In addition, homologs of two other hits have been shown to modulate the toxicity of oligomeric A. The PBS2 homolog MAP2K4, part of the JNK pathway, is activated in response to Aβ oligomer administration to cortical neuron cultures (Bozyczko-Coyne, D. et al. J Neurochem (2001) 77:849). Synaptojanin, the INP52 homolog, has also been shown to modulate the toxicity of Aβ oligomers (Berman et al. (2008) Nat Neurosci 11, 547).

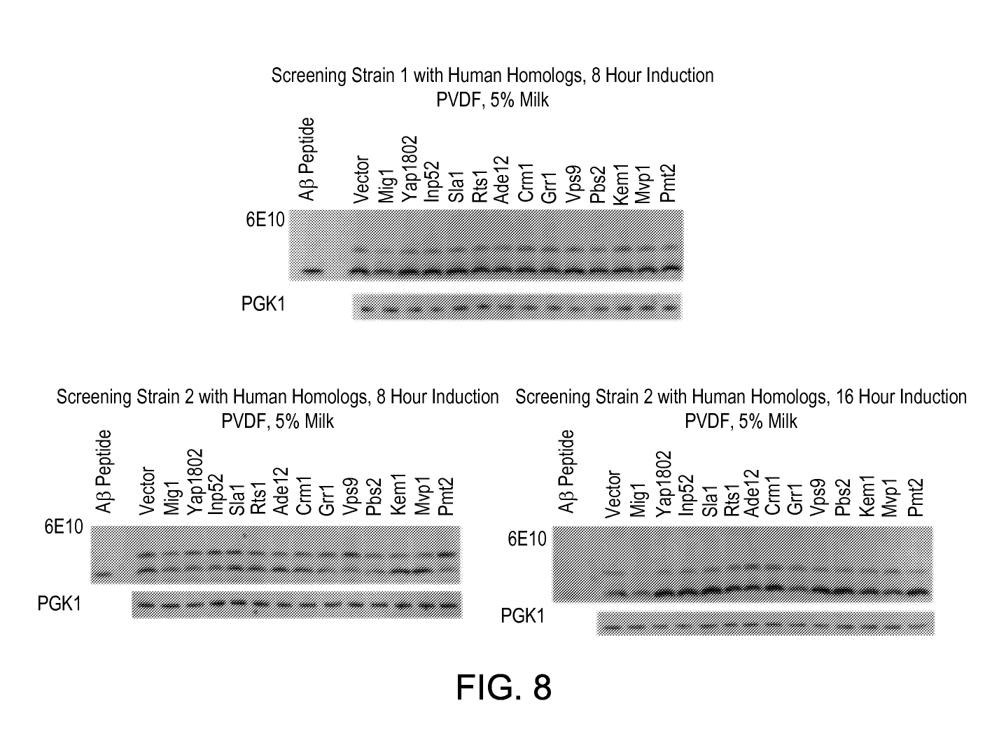

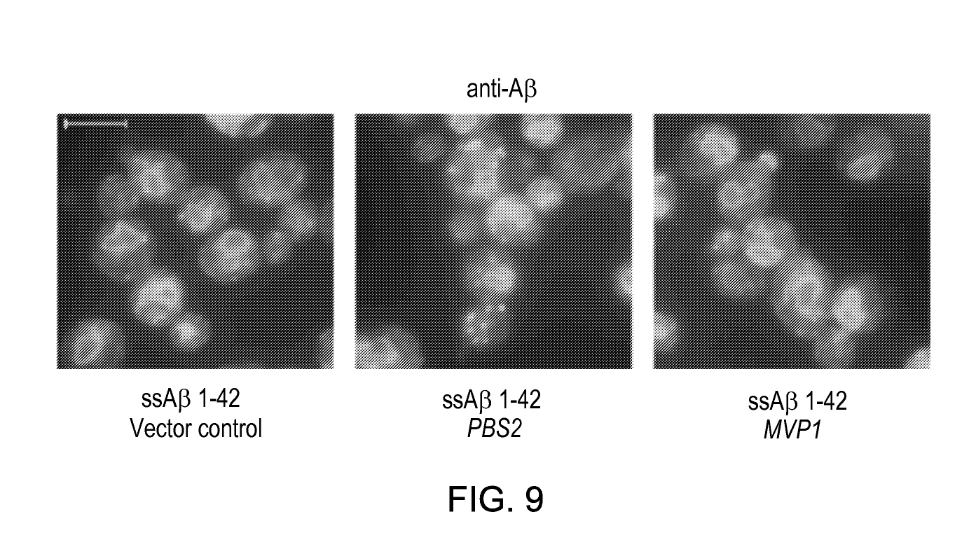

Overall, results suggest that the screen hits with clear human homologs do not alter Aβ levels significantly (FIG. 8). They also have little effect on Aβ localization; only the MAP2K4 homolog PBS2 enhances the formation of Aβ foci (FIG. 9). Thus the screen hits appear to modulate cellular pathways required for coping with Aβ toxicity rather than decreasing it through Aβ clearance.

### Example 5

### Effect of Aβ on Endocytosis and Vesicular Trafficking in Yeast

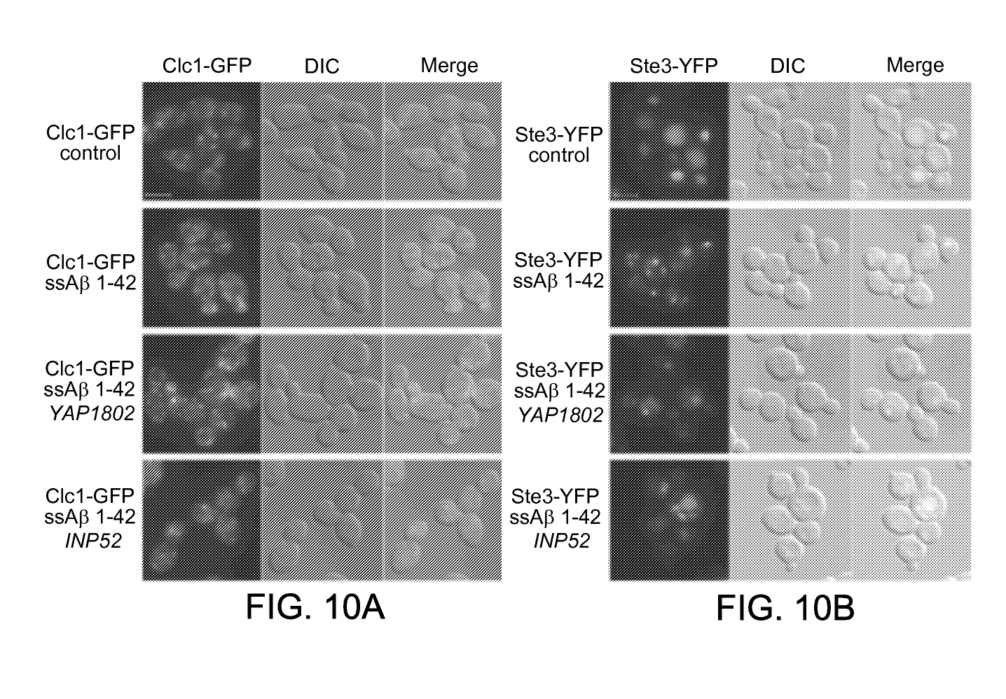

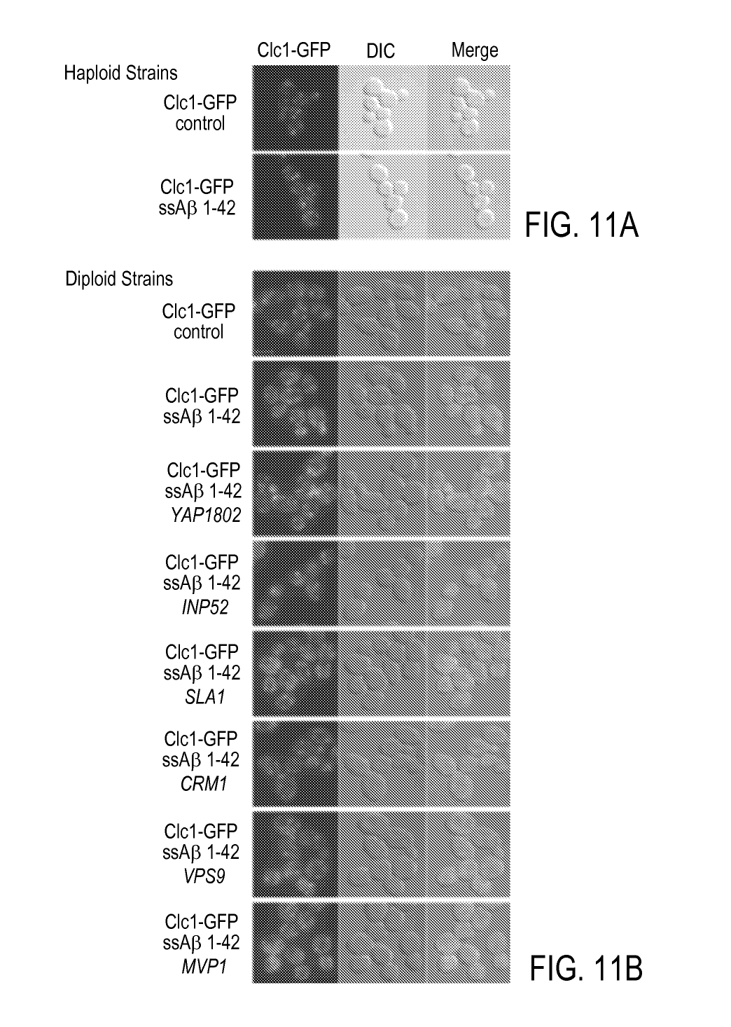

Our genetic hits suggest that clathrin-mediated endocytosis plays a role in modulating Aβ 1-42 toxicity. To investigate whether Aβ negatively affects endocytosis per se and/or whether endocytosis simply helps to shunt Aβ into compartments where it is less toxic, we monitored endocytic trafficking in a yeast strain in which clathrin light chain was endogenously tagged with GFP(Clcl-GFP). As expected, the control strain exhibited only a few faint foci of Clcl-GFP (Sun, S. et al. (2007) J Cell Biol 177: 355). Expression of ssAβ 1-42 increased the number and brightness of foci, and decreased their size (FIG. 10A & FIG. 11A). The small foci of Clcl-GFP in the ssAβ 1-42 expressing strain likely represent accumulated endocytic intermediates. Thus, our findings suggest that Aβ affects clathrin-mediated endocytosis.

To test the effects of Aβ on the trafficking of an endocytosis substrate we examined a Ste3-YFP fusion. This mating pheromone receptor is constitutively targeted to the plasma membrane. In the absence of its ligand, it is then endocytosed and degraded in the vacuole (Maldonado-Baez et al., (2008) Mol Biol Cell 19, 2936). Ste3-YFP was primarily localized to the vacuole in the control strain. In contrast, in an ssAβ 1-42 expressing strain Ste3-YFP was found in foci surrounding the vacuole establishing that Aβ caused a defect in the trafficking of this substrate (FIG. 10B).

### Example 6

### Aβ Toxicity Modifiers Modulate the Aβ-Induced Change in Clathrin Localization

Next, we examined the effects of genetic hits involved in endocytosis and trafficking. YAP1802 promotes clathrin assembly (Maldonado, et al, supra) and indeed, it increased the number of clathrin foci in the Aβ strain (FIGS. 10A and 11A). INP52, on the other hand, facilitates fission and uncoating of clathrin vesicles (Cohen, et al., (2006) Science 313: 1604). It largely reversed the effect of Aβ 1-42 (FIG. 11B). While the genes have different effects on clathrin assembly, both should act to increase flux through the endocytic pathway.

We expressed ssAβ 1-42 in strain carrying a haploid clathrin light chain GFP fusion (Clcl-GFP). ssAβ 1-42 expression resulted in an increase in the number and brightness of Clcl-GFP foci, while also decreasing their size. Since this GFP-fusion strain, part of the GFP library, is incompatible with the ssAβ 1-42 integration construct, we used a GAL-driven multi copy vector. Next, we examined the effects of suppressors and enhancers with annotated functions in endocytosis and trafficking on this Aβ-induced phenotype. To achieve robust Aβ toxicity in the Clcl-GFP background, we mated one of the ssAβ 1-42 screening strains to the Clcl-GFP strain. While Aβ toxicity was attenuated in the diploid, it still induced a similar change in Clcl-GFP localization. We then tested the effects of the modifiers in this background. Clcl-GFP foci became more plentiful, brighter and smaller as a result of expression of YAP1802, which is involved in clathrin cage assembly (FIG. 11B). In contrast, INP52, involved in clathrin vesicle fission and recycling (Stefan, et al. (2005). Mol Cell Biol 25: 2910), had the opposite effect. Without wishing to be bound by any theory, the Clcl-GFP foci likely represent endocytic intermediates that accumulate upon ssAβ 1-42 expression. Yap1802p counteracts this defect by increasing vesicle formation, while Inp52p acts to complete the fission of otherwise stalled vesicles. As such both proteins result in an increased flux through the endocytic pathway.

### Example 7

### Aβ Toxicity Modifiers Modulate the Aβ-Induced Change in Ste3-YFP Trafficking

To examine the effect of Aβ on endocytosis and protein trafficking, we created our own version of the Ste3 localization assay described in Maldonado, supra). The mating pheromone receptor Ste3p is constitutively targeted to the plasma membrane, but in the absence of its ligand it is endocytosed and degraded in the vacuole. We generated a construct encoding Ste3-YFP under the control of the constitutive GPD promoter and integrated it into a control and an ssAβ 1-42 screening strain. ssAβ 1-42 expression changed Ste3-YFP localization from mostly vacuolar to cytoplasmic foci surrounding the vacuole, clearly showing that Aβ causes a defect in Ste3-YFP trafficking (FIG. 10B).

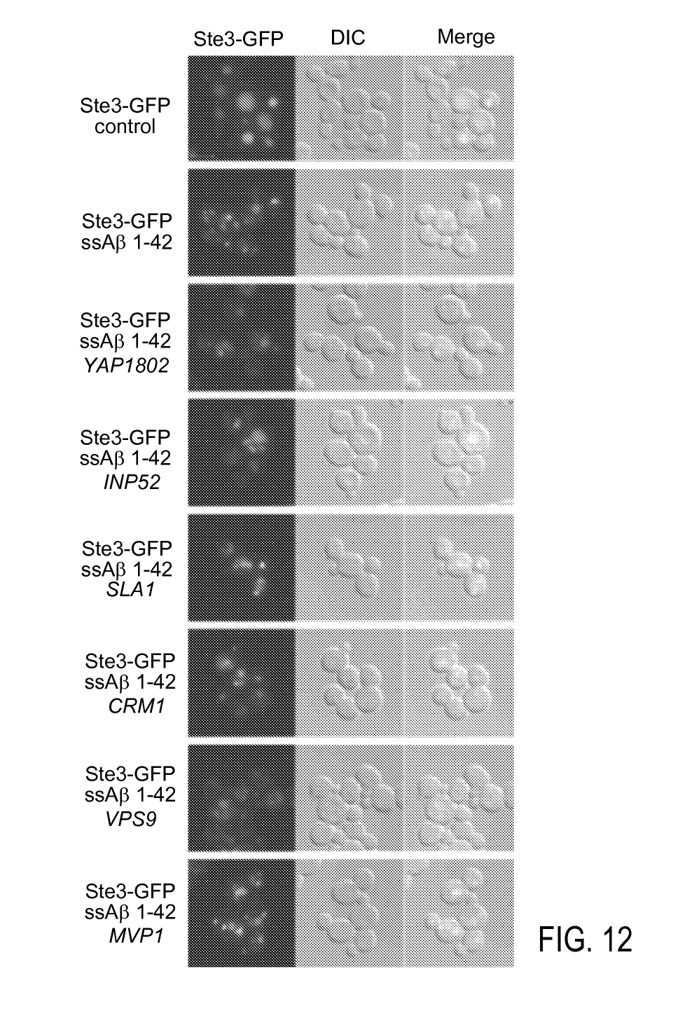

We then examined the effects of modifiers involved in endocytosis and protein trafficking. The endocytic genes YAP1802, INP52 and SLA1 reversed the effect induced by Aβ (FIGS. 10B and 12). Sla1p functions, as Yap1802p, in clathrin assembly. As such these endocytic suppressors reversed the effect of Aβ by increasing endocytic trafficking. The suppressor VPS9 appeared to slightly reverse the effect of Aβ as well, but also changed vacuolar morphology and reduced the amount of Ste3-YFP fluorescence. Expression of the enhancer MVP1 slightly enhanced the Aβ-induced defect in Ste3-YFP trafficking; the strain accumulated more Ste3-YFP foci.

### Materials and Methods

### Further Details Regarding Constructs, Strains and Growth Conditions

The ssAβ1-42 construct consists of attB sites for Gateway cloning the Kar2 signal sequence and the Aβ1-42 sequence. The Aβ sequence was codon optimized for expression in yeast. The entire construct was synthesized and cloned into the Gateway entry vector pDONR221.

The sequence of the ssAβ construct (SEQ ID NO: 2) is shown above. It will be appreciated that the sequence contains Gateway flanking regions at the 5′ and 3′ ends (nucleotides 1-32 and 288-312, respectively).

The same approach was used to generate the ssAβ1-40 construct. The BPTI WT and C51A constructs were the kind gift of Dane Wittrup(7). The original BPTI construct contains a signal sequence, but we replaced it with the Kar2 signal sequence in order to target them in the same manner as Aβ. The Kar2ss sequence and Gateway flanking regions were added to the BPTI ORFs using overlap extension PCR. The Pdi1 gene is part of the overexpression library used in the screen. The Pdi1 gene was gateway cloned into the pDONR221 entry vector. The Aβ and BPTI constructs as well as Pdi1 were cloned into the pAG Gal p426 vector (8). Constructs were transformed into W303 Mat α, can1-100, his3-11,15, leu2-3,112, trp1-1, ura3-1, ade2-1 using a standard lithium acetate transformation protocol.

To generate ssAfβ1-42 screening strains the ssAβ1-42 construct was moved to a pAG Gal p305 expression vector (8). The plasmid was digested using BstX1, gel purified and transformed into W303. The transformation was carried out in duplicate and the level of growth of 16 transformants each was tested on synthetic deficient media lacking leucine with galactose. Two strains from the independent transformations where chosen as screening strains based on their robust yet intermediate toxicity that would allow for the identification of both suppressors and enhancers. Several transformants that showed no toxicity were chosen as 1×ssAβ controls. The control strain for wild type yeast growth is carrying a Gal inducible YFP integrated in the same fashion as the ssAfβ1-42 constructs. For spotting assays strains were grown over night at 30° C. in 3 ml SD media lacking the relevant amino acids and containing glucose. Cell concentrations (OD600) were adjusted in a 96-well plate to that of the strain with the lowest concentration. Cells were then 5 fold serially diluted and spotted on SD media containing glucose (Uninduced) and galactose (Induced). Plates were incubated at 30° C. for 2 (glucose) or 3 days (galactose).

### Cell Lysis and Western Analysis

Strains were grown in synthetic deficient media lacking leucine and uracil (SD-Leu-Ura) with raffinose overnight at 30° C. Cultures were the diluted into inducing media containing galactose (OD600 0.2) and grown for 8 h. Cells were spun down for 5 min at 3,000 rpm. The supernatant was removed and the cells were resuspended in 200 ul lysis buffer (50 mM Hepes pH7.5, 150 mM NaCl, 2.5 mM EDTA, 1% Triton X-100, 50 mM NEM, 1 mM PMSF, 1 tablet Roche Complete Mini EDTA-free protease inhibitor cocktail/5 ml). Cells were kept on ice from this point on. Cells were transferred to an eppendorf tube containing ˜200 ul glass beads and lysed by shaking for 3 min on a bead beater. Cell lysates were collected by puncturing the bottom of the eppendorf tube with a 20 gauge needle, sticking the punctured tube into a fresh tube and spinning them in a benchtop centrifuge at 6000 rpm for 15 seconds. 150 ul of cell lysate were collected and transferred to a new tube. Protein concentrations were measured using a BCA Bradford assay and equalized to the levels of the sample with the lowest concentration. 100 ul of the equalized lysates were then mixed with 2×SDS sample buffer (Laemmli buffer) and boiled for 5 min. Samples were run on Invitrogen NuPage Novex 4-12% Bis-Tris gels using MES SDS running buffer. Proteins were transferred to PVDF using a semi-dry transfer apparatus. For blots shown in FIG. 2, membranes were blocked with 5% milk 1×PBS, and subsequently probed with the mouse monoclonal anti-Aβ antibody 6E10 (Signet laboratories) at a dilution of 1:1,000. Blots were subsequently reacted with the antimouse IgG peroxidase conjugate (Sigma) at a dilution of 1:1,000. Blots were developed with either Super Signal West Pico Chemiluminescent Substrate (Thermo Scientific) or with Immobilon Western Chemiluminescent HRP Substrate (Millipore). For the loading control, blots were probed with a mouse monoclonal anti-PGK1 antibody at a dilution of 1:2,000 (Molecular Probes). For the Western Blots showing the effect of the human homologs on Ab levels, membranes were blocked with 5% milk 1×PBS, probed with the mouse monoclonal anti-Aβ antibody 6E10 (Signet laboratories) at a dilution of 1:1,000. Blots were subsequently reacted with the anti-mouse secondary antibody DyLight 800 (Rockland Immunochemicals) at 1:10,000. For the loading control, blots were probed with a mouse monoclonal anti-PGK1 antibody at a dilution of 1:2,000 (Molecular Probes). Blots were scanned using an Odyssey scanner (L1-COR Biosciences).

### Immunostaining

Strains were pregrown in raffinose media overnight and then induced in galactose media for 8 hours (5 ml OD600 0.2). Cells were spun down and resuspended in 1 ml 3.7% formaldehyde (37% formaldehyde in 0.1M KPi (potassium phosphate buffer) pH6.4) after removal of supernatant. Cells were fixed over night at 4° C. After the fixation cells were washed three time in 1 ml 0.1M KPi pH 6.4 and then resuspended in 1 ml 1.2M sorbitol-citrate buffer (1L: 218.6 g sorbitol, 17.40 g anyhydrorus K2HPO4, 7 g citric acid; filter sterilize). Cells were spun down again and resuspended in 200 ul of digestion mix (200 ul 1.2M sorbitol-citrate, 20 ul glusolase and 2 ul 10 mg/ml zymolase). Cells were incubated in the digestion mix for 45 min at 30° C. During the incubation 5 ul 0.1% polylysine was added to each well of a 30 well slide (Thermo ER-212W). After 5 min of incubation the slides was washed with distilled water and allowed to air dry completely. Digested cells were spun down at 3,000 rpm for 3 min and gently resuspended in 1 ml sorbitol-citrate. Cells were spun down again and then resuspended in a volume of sorbitol citrate dependent on cell pellet size (15-50 ul). 5 ul of cells was added to each well and incubated for 10 min. Cells were removed from the side of the well using a vacuum tip. If the cell density was low, as revealed by light microscopy, more cells were added. The slides were then incubated in ice-cold methanol for 3 min, followed by 10 sec in ice-cold acetone. Acetone was shook off and slides air-dried. As the primary antibody 4 ul of 1:200 6E10 in PBS/BSA (1% BSA, 0.04M K2HPO4, 0.01M KH2PO4, 0.15M NaCl, 0.1% NaN3; for 100 ml: 1 g BSA, 4 ml 1M K2HPO4, 1 ml 1M KH2PO4, 15 ml 1M NaCl, 1 ml 10% NaN3, sterilized water to 100 ml) were added to each well. Slide was incubated over night at room temperature in a wet chamber. After the incubation the antibody was removed using a vacuum tip and each well was washed 3 times with PBS/BSA. Then 4 ul of the secondary antibody, 1:100 anti-mouse FITC, was added to each well and incubated for 2 hours. Subsequently, each well was washed 4 times with PBS/BSA. 1 ul of DAPIIMOUNT was added to each well prior to adding the coverslip and sealing the slide with nail polish. For co-immunostaining cells were processed as described above but incubated with 1:200 6E10 and 1:100 rabbit anti-GFP in PBS/BSA and subsequently 1:100 antimouse Alexa Fluor 594 and 1:100 anti-rabbit Alexa Fluor 488 in PBS/BSA simultaneously. The GFP-tagged strains are part of the yeast GFP library(4). Images were taken on a Zeiss Aviovert. Final images were assembled from the different channels (GFP, DAPI and dsRed) in Adobe Photoshop. Brightness and contrast were adjusted equally for all images.

### Clioquinol Rescue

Yeast strains were grown overnight to saturation in synthetic medium with glucose as the carbon source after inoculation from a colony. These cultures were used to inoculate raffinose cultures. Raffinose cultures in log phase growth (OD600 0.7-0.9) were diluted to an OD600 of 0.03 in synthetic medium with galactose as the carbon source, and 300 ml of the cell suspension was distributed to each well of a Bioscreen plate. 3.03 ml of clioquinol (Sigma C8133, ˜95%), dissolved in DMSO at 100 times the final intended concentration, was added to each well. A Labsystems Bioscreen C was used to maintain the plate at 30 degrees C. and make OD600 measurements on each well at 10-minute intervals over a period of approximately three days. Each condition was performed in duplicate. The most effective concentration of clioquinol is slightly variable. In our experiments the most effective concentration varied from 0.5 to 0.8 uM.

### Screen for Modifiers of Aβ Toxicity

The overexpression library screened contains ˜5800 full-length sequence verified yeast ORFs in the galactose-inducible Gateway expression plasmid pBY011 (CEN, URA3, AmpR) (9). The library is arrayed in 96-well format. Plasmid DNA was prepared by pin inoculation into deep well 96-well plates containing 1.8 ml LB-AMP, growth over night at 37° C. and 96-well mini preps using a Qiagen BioRobot 8000. The DNA was transformed into a ssAβ screening strain (ssAβ1-42 p305) carrying a Gal4-ER-VP16 plasmid (CEN, HIS3, AmpR), which allows for expression from GAL promoters on carbon sources other than galactose in the presence of estradiol in the yeast media(3). Neither estradiol nor the Gal4-ER-VP16 plasmid had an effect on Aβ toxicity on their own. Transformations were carried out using a standard lithium acetate transformation protocol adapted for a 96-well format and automation using a Tecan Evo 150 liquid handling robot. Transformants were grown in synthetic deficient media lacking histidine, leucine, and uracil (SD-His-Leu-Ura) with glusose overnight. The cells were then diluted in water and spotted on SD-His-Leu-Ura agar plates containing glucose alone (control), galactose alone, glucose plus 1 uM estradiol (Sigma E1024) or glycerol plus 1 uM estradiol using a Singer RoToR pinning robot and long 96-well pins. Putative enhancers and suppressors were identified after 2-4 days of growth at 30° C. Putative screen hits were cherry picked from the plasmid library, retransformed into two independent derived ssAβ screening strains and retested on the three screening conditions in two independently derived strains. We eliminated hits that have known effects on GAL induction and genes whose overexpression has previously been shown to be toxic. To further exclude false-positive suppressors we used flow cytometry to measure the expression of YFP in their presence. To further exclude false-positive enhancers that cause a general inhibition when overexpressed we examined their effects in the YFP control strain, which has no growth impairment. The identity of confirmed modifiers was verified by sequencing.

### Flow Cytometry

A strain carrying an integrated YFP was transformed with the putative Aβ suppressors. The resulting strains were grown in glucose media in a 96 well format, diluted into the various inducing media (galactose, glucose+1 uM estradiol, glycerol+1 uM estradiol) [5 ul culture added to 120 ul media], and incubated over night at 30° C. with mild shaking. These overnight cultures were diluted 20 fold into water and YFP levels were measured using a Guava flow cytometer. Each strain was measured 3 times and 5000 cells were counted for each well. The whole experiment was repeated 3 times. Values are averages of these 3 experiments and reported in percent of the vector control strain YFP levels.

### Microscopy of Clcl-GFP and Ste3-YFP

To examine the effects of Aβ on endocytosis we used the clathrin light chain (Clcl)-GFP strain from the GFP library (4). In experiments to examine the effects of Aβ alone we transformed the GFP-fusion strain with the ssAβ 1-42 construct on a multi copy vector and the corresponding vector control. For all microscopy experiments strain were precultured in raffinose media and then induced in galactose media. We also used GFP fusion strains of other endocytic proteins (Abp1, Sla1 and Sla2) and observed similar Aβ-induced changes in localization as with Clcl-GFP (data not shown); yet the fluorescence of these fusions was rather low. To test the effects of screen hits on the Clcl-GFP Aβ phenotype, we mated the Clcl-GFP fusion strain to a set of Aβ screening strains carrying individual modifiers. We used this approach as expression of two genes from plasmids, one with varying copy number, can lead to inconsistent results when examining individual cells; as is the case for microscopy. Furthermore, the GFP-fusion strains on their own are incompatible with integration constructs due to their complete deletion of auxotrophic markers. While Aβ toxicity was reduced in the resulting diploid, ssAβ 1-42 expression resulted in the same Clcl-GFP phenotype.

We created our own version of the Ste3 localization assay (5) by generating a GPD-driven Ste3-YFP construct, using the Ste3 plasmid from our ORF library and a GPD p303 vector from the pAG collection (8), and integrating it into an ssAβ 1-42 screening strain as well as a control strain. We tested the effect of selected modifiers by transforming these strains with the modifiers and analyzing them in the same fashion. For both assays, we found that modifiers alone had no effect on localization. Expression was induced for 16 h and GFP or YFP fluorescence monitored using a Zeiss Axiovert microscope. Brightness and contrast were adjusted equally for all images.

### REFERENCES FOR MATERIALS AND METHODS

1. G. E. Gibson, Q. Shi, A mitocentric view of Alzheimer's disease suggests multifaceted treatments. J Alzheimers Dis 20 Suppl 2, S591 (2010).
2. J. X. Chen, S. S. Yan, Role of mitochondrial amyloid-beta in Alzheimer's disease. J Alzheimers Dis 20 Suppl 2, S569 (2010).
3. M. J. Quintero, D. Maya, M. Arevalo-Rodriguez, A. Cebolla, S. Chavez, An improved system for estradiol-dependent regulation of gene expression in yeast. Microb Cell Fact 6, 10 (2007).
4. R. Howson et al., Construction, verification and experimental use of two epitope tagged collections of budding yeast strains. Comp Funct Genomics 6, 2 (2005).
5. L. Maldonado-Baez et al., Interaction between Epsin/Yap180 adaptors and the scaffolds Ede1/Pan1 is required for endocytosis. Mol Biol Cell 19, 2936 (2008).
6. C. J. Stefan, S. M. Padilla, A. Audhya, S. D. Emr, The phosphoinositide phosphatase Sj12 is recruited to cortical actin patches in the control of vesicle formation and fission during endocytosis. Mol Cell Biol 25, 2910 (2005).
7. J. M. Kowalski, R. N. Parekh, K. D. Wittrup, Secretion efficiency in Saccharomyces cerevisiae of bovine pancreatic trypsin inhibitor mutants lacking disulfide bonds is correlated with thermodynamic stability. Biochemistry 37, 1264 (1998).
8. S. Alberti, A. D. Gitler, S. Lindquist, A suite of Gateway cloning vectors for highthroughput genetic analysis in Saccharomyces cerevisiae. Yeast 24, 913 (2007).
9. T. F. Outeiro, S. Lindquist, Yeast cells provide insight into alpha-synuclein biology and pathobiology. Science 302, 1772 (2003).

### OTHER EMBODIMENTS

It is to be understood that, while the invention has been described in conjunction with the detailed description thereof, the foregoing description is intended to illustrate and not limit the scope of the invention. It is also to be understood that claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process.

The invention also includes embodiments in which more than one, or all of the group members are present in, employed in, or otherwise relevant to a given product or process. Furthermore, it is to be understood that the invention encompasses all embodiments in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise and such embodiments do not constitute added matter or extend beyond the content of the application as filed. Where elements are presented as lists, it is to be understood that each subgroup of the elements is also disclosed, and any one or more element(s) can be removed from the group, and such subgroup or resulting list is explicitly disclosed herein and does not constitute added matter or extend beyond the content of the application as filed. It should it be understood that, in general, where the invention, or aspects of the invention, is/are referred to as comprising particular elements, features, etc., certain embodiments of the invention or aspects of the invention consist, or consist essentially of, such elements, features, etc. It should also be understood that any embodiment of the invention, can be explicitly excluded from the claims.

Other aspects, advantages, and modifications of the invention are within the scope of the claims set forth below.

What is claimed is:

1-29. (canceled)

30. A method of modulating amyloid beta-mediated toxicity, the method comprising contacting a cell expressing a toxicity-inducing amount or form of amyloid beta with an effective amount of a compound that modulates expression or activity of SPO7, KAR9, POG1, KEM1, XRN1, ROM1, NET1, MID2, BOP3, PMT2, POMT2, PSK1, PASK, YBL061C, SEL1L2, PET111, SLS1, SVL3, IVY1, MVP1, SNX8, PBS2, MAP2K4, PKC1, PKN2, WHI5, SLF1, LARP1, YBL086C, YAP1802, PICALM, YPL014W, RTS1, PPP2R5C, SPT21, FMP48, MARK4, PPR1, TEC1, TEAD2, ADE12, ADSSL1, CRM1, XPO1, NAB3, SLAT, SH3KBP1, RTG3, MITF, SRO9, LARP1B, MBP1, DAPK1, MUM2, INP52, SYNJ1, FCY21, GRR1, FBXL2, VPS9, RABGEF1, or OPY1.

31. The method of claim 30, wherein the method comprises contacting a cell expressing a toxicity-inducing amount or form of amyloid beta with an effective amount of a compound that modulates expression or activity of PICALM, PPP2R5C, ADSSL1, XPO1, SH3KBP1, SYNJ1, FBXL2, RABGEF1, MARK4, TEAD2, MITF, DAPK1, LARP1, LARP1B, XRN1, POMT2, SNX8, MAP2K4, PKN2, PASK, NET1, or SEL1L2.

32. The method of claim 30, wherein the method comprises contacting a cell expressing a toxicity-inducing amount or form of amyloid beta with an effective amount of a compound that modulates expression or activity of SPO7, KAR9, POG1, KEM1, XRN1, ROM1, NET1, MID2, BOP3, PMT2, POMT2, PSK1, PASK, YBL061C, SEL1L2, PET111, SLS1, SVL3, IVY1, MVP1, SNX8, PBS2, MAP2K4, or PKC1.

33. The method of claim 30, wherein the method comprises contacting a cell expressing a toxicity-inducing amount or form of amyloid beta with an effective amount of a compound that inhibits expression or activity of SPO7, KAR9, POG1, KEM1, XRN1, ROM1, NET1, MID2, BOP3, PMT2, POMT2, PSK1, PASK, YBL061C, SEL1L2, PET111, SLS1, SVL3, IVY1, MVP1, SNX8, PBS2, MAP2K4, or PKC1.

34-47. (canceled)

48. A method of identifying a compound that inhibits or increases expression or activity of a protein, the method comprising: providing a cell expressing PICALM, PPP2R5C, ADSSL1, XPO1, SH3KBP1, SYNJ1, FBXL2, RABGEF1, MARK4, TEAD2, MITF, DAPK1, LARP1, LARP1B, XRN1, POMT2, SNX8, MAP2K4, PASK, NET1, SEL1L2, or PKN2 protein; contacting the cell with an agent; and measuring the expression or activity of the protein in the presence of the agent, wherein a reduction or increase in the expression or activity of the protein in the presence of the agent as compared to the expression or activity of the protein in the absence of the agent identifies the agent as a compound that inhibits or increases, respectively, the expression or activity of the protein.

49. The method of claim 48, wherein the screening is to identify an agent that inhibits expression or activity of XRN1, POMT2, SNX8, MAP2K4, PASK, NET1, SEL1L2, or PKN2.

50. The method of claim 48, wherein the screening is to identify an agent that increases expression or activity of PICALM, PPP2R5C, ADSSL1, XPO1, SH3KBP1, SYNJ1, FBXL2, RABGEF1, MARK4, TEAD2, MITF, DAPK1, LARP1, or LARP1B.

51-56. (canceled)

57. A method of identifying a compound that inhibits amyloid beta-mediated toxicity, the method comprising: (a) providing a cell expressing an amount or form of amyloid beta that reduces viability of the cell; (b) contacting the cell with an agent that modulates expression or activity of SPO7, KAR9, POG1, KEM1, XRN1, ROM1, NET1, MID2, BOP3, PMT2, POMT2, PSK1, PASK, YBL061C, SEL1L2, PET111, SLS1, SVL3, IVY1, MVP1, SNX8, PBS2, MAP2K4, PKC1, PKN2, WHI5, SLF1, LARP1, YBL086C, YAP1802, PICALM, YPL014W, RTS1, PPP2R5C, SPT21, FMP48, MARK4, PPR1, TEC1, TEAD2, ADE12, ADSSL1, CRM1, XPO1, NABS, SLA1, SH3KBP1, RTG3, MITF, SRO9, LARP1B, MBP1, DAPK1, MUM2, INP52, SYNJ1, FCY21, GRR1, FBXL2, VPS9, RABGEF1, or OPY1; and (c) measuring cell viability in the presence of the agent, wherein an increase in cell viability in the presence of the agent as compared to cell viability in the absence of the agent identifies the agent as a compound that inhibits alpha synuclein-mediated toxicity.

58. The method of claim 57, wherein the agent modulates expression or activity of KEM1, XRN1, PMT2, POMT2, MVP1, SNX8, PBS2, MAP2K4, YAP1802, PICALM, RTS1, PPP2R5C, ADE12, ADSSL1, CRM1, XPO1, SLA1, SH3KBP1, INP52, SYNJ1, GRR1, FBXL2, VPS9, or RABGEF1.

59. The method of claim 57, wherein the agent inhibits expression or activity of SPO7, KAR9, POG1, KEM1, XRN1, ROM1, MID2, BOP3, PMT2, POMT2, PSK1, YBL061C, PET111, SLS1, SVL3, IVY1, MVP1, SNX8, PBS2, MAP2K4, or PKC1.

60. The method of claim 57, wherein the agent inhibits the expression or activity of KEM1, XRN1, PMT2, POMT2, MVP1, SNX8, PBS2, or MAP2K4.

61-65. (canceled)

66. The method of claim 48, wherein the cell is a yeast cell.

67. The method of claim 48, wherein the cell is a mammalian cell.

68-84. (canceled)

85. A cell comprising: (a) a first heterologous nucleic acid encoding a toxicity-inducing form of amyloid beta; and (b) a second heterologous nucleic acid encoding SPO7, KAR9, POG1, KEM1, XRN1, ROM1, NET1, MID2, BOPS, PMT2, POMT2, PSK1, PASK, YBL061C, SEL1L2, PET111, SLS1, SVL3, IVY1, MVP1, SNX8, PBS2, MAP2K4, PKC1, PKN2, WHI5, SLF1, LARP1, YBL086C, YAP1802, PICALM, YPL014W, RTS1, PPP2R5C, SPT21, FMP48, MARK4, PPR1, TEC1, TEAD2, ADE12, ADSSL1, CRM1, XPO1, NAB3, SLA1, SH3KBP1, RTG3, MITF, SRO9, LARP1B, MBP1, DAPK1, MUM2, INP52, SYNE, FCY21, GRR1, FBXL2, VPS9, RABGEF1, or OPY1.

86. The cell of claim 85, wherein the second heterologous nucleic acid encodes WHI5, SLF1, LARP1, YBL086C, YAP1802, PICALM, YPL014W, RTS1, PPP2R5C, SPT21, FMP48, MARK4, PPR1, TEC1, TEAD2, ADE12, ADSSL1, CRM1, XPO1, NAB3, SLA1, SH3KBP1, RTG3, MITF, SRO9, LARP1B, MBP1, DAPK1, MUM2, INP52, SYNE, FCY21, GRR1, FBXL2, VPS9, RABGEF1, or OPY1.

87. (canceled)

88. The cell of claim 85, wherein the first heterologous nucleic acid, the second heterologous nucleic acid, or both, are integrated into the genome of the cell.

89. The cell of claim 85, wherein the first heterologous nucleic acid, the second heterologous nucleic acid, or both, are operably linked to inducible promoter(s).

90-92. (canceled)

93. The cell of claim 85, wherein the cell is a yeast cell.

94. The cell of claim 85, wherein the gene is a mammalian gene and the cell is a mammalian cell.

95. The cell of claim 85, wherein the gene is a human gene and the cell is a human cell.


SEQUENCE LISTING (3 sequences)

SEQ ID NO: 1 - protein, 84 aa, Artificial Sequence
  fusion protein
MFFNRLSAGKLLVPLSVVLYALFVVILPLQNSFHSSNVLVRGDAEFRHDSGYEVHHQKLVFFAEDVGSNKGAIIGLMVGGVVIA

SEQ ID NO: 2 - DNA, 312 bp, Artificial Sequence
  fusion protein expression construct
acaagtttgtacaaaaaagcaggcttcacaaaatgtttttcaacagactaagcgctggcaagctgctggtaccactctccgtggtcctgtacgcccttttcgtggtaatattacctttacagaattctttccactcctccaatgttttagttagaggtgatgctgaatttagacatgattctggttatgaagttcatcatcaaaaattggttttttttgctgaagatgttggttctaataaaggtgctattattggtttgatggttggtggtgttgtcattgcttaaacccagctttcttgtacaaagtggt

SEQ ID NO: 3 - protein, 43 aa, homo sapian
DAEFRHDSGYEVHHQKLVFFAEDVGSNKGAIIGLMVGGVVIAT